(19) United States
(12) Patent Application Publication
(10) Pub. No.: US 2025/0134153 A1
(43) Pub. Date: May 1, 2025

(54) Mycelium-Based Encapsulation of Fats and/or oils for Controlled Release in Foods and Cosmetics

(71) Applicant: Optimized Foods, Inc., Davis, CA (US)

(72) Inventors: Lynn Hanson, Fairfield, CA (US); Zane Starkewolfe, Davis, CA (US); Cody Yothers, Davis, CA (US)

(73) Assignee: Optimized Foods, Inc., Davis, CA (US)

(21) Appl. No.: 18/935,371

(22) Filed: Nov. 1, 2024

Related U.S. Application Data
(60) Provisional application No. 63/594,958, filed on Nov. 1, 2023.

(51) Int. Cl. A23P 10/30; A23L 29/00; A61K 8/11; A61K 8/9728

(52) U.S. Cl. CPC A23P 10/30; A23L 29/065; A61K 8/11; A61K 8/9728

(57) ABSTRACT

Novel fungal food and cosmetic products and methods of creating them are disclosed with the products having various textures, flavors, and biologic activities. These fungal foods and cosmetic products have controlled nutrient release. An exemplary process uses fungal biomass fat and/or oil encapsulation to control the texture, flavor, nutrient characteristics, product performance characteristics and nutrient release. The fat and/or oil encapsulation can be used with other methods to create a wide array of products with novel flavors, textures, and nutrient characteristics, and the products can be modified depending on the intended application.

The present invention claims priority under 35 USC 119(e) to U.S. Provisional Application No. 63/594,958 filed Nov. 1, 2023, the entire contents of which are incorporated by reference in its entirety.

### FIELD OF THE INVENTION

In its broadest sense, the present invention relates to modified fungal pellets and their uses. The present invention relates to novel fungal food and cosmetic products and methods of creating them. The products having various textures, flavors, and biologic activities and the fungal foods and cosmetic products have controlled nutrient release. In an embodiment, fungal biomass fat and/or oil encapsulation are used to control the texture, flavor, nutrient characteristics, product performance characteristics and the nutrient release. Fat and/or oil encapsulation can be used with other methods to create a wide array of products with novel flavors, textures, and nutrient characteristics. Moreover, the products can be modified as described herein depending on the intended application.

### BACKGROUND OF THE INVENTION

Fats and/or oils are integral components in a wide range of food and cosmetic products, contributing to their structure, taste, texture, and other sensory properties. Fats and/or oils are derived from various sources, including plants, fungi, animals as well as by synthetic means. In recent years, there has been a growing interest in alternative sources of fats, such as plant-based, cell-cultured and synthetic fats, due to environmental, health, and ethical considerations.

The world of food and cosmetic products relies heavily on fats and oils as fundamental building blocks for taste, texture, and structural integrity. These components contribute not only to sensory properties but also serve as key elements in the formulation of countless consumer goods. Traditionally, these fats and oils have been sourced from various origins, including plants, animals, fungi, and synthetic processes. However, recent years have seen a significant shift in the industry, driven by increasing concerns over environmental sustainability, health implications, and ethical considerations. Replacing a commonly used fat or oil with alterative fats and oils poses the dilemma of requiring the same properties of the commonly used fat or oil from an alternative. This has given rise to a pressing need for innovative fat delivery platforms, especially within the realm of alternative plant-based foods and cosmetics applications, to which this invention applies.

The shift towards plant-based and alternative fat sources has been notably propelled by mounting concerns regarding environmental sustainability. The conventional methods of fat production, often reliant on animal agriculture and resource-intensive agricultural practices, contribute to deforestation, greenhouse gas emissions, and depletion of natural resources. Moreover, there's a growing demand for food products that align with health-conscious consumer preferences, such as reduced saturated fats, cholesterol, and allergenic components. As such, innovative fat delivery and/or encapsulation systems are paramount in addressing these concerns, enabling the formulation of plant-based and alternative food products that are not only eco-friendly but also meet rigorous health standards.

Furthermore, the ethical dimension plays an increasingly influential role in consumer choices. Plant-based fats and oils, as well as synthetic alternatives, have garnered attention due to their alignment with animal welfare concerns and the need for cruelty-free products. The surge in interest in fats such as cell-cultured fats and the desire to work with fats that are less environmentally detrimental (e.g., avoiding palm and coconut oils) are aspects of this ethical shift, as it offers the potential to eliminate the need for traditional animal farming altogether. As the legal landscape evolves to protect these advancements, it is clear that innovative fat delivery platforms for alternative plant-based foods, hybrid foods, and novel foods from microbial or other sources are important in meeting the diverse demands of a changing market.

In the cosmetics industry, the demand for innovative fat delivery and/or encapsulation platforms is equally compelling.

Fats and oils serve as foundational ingredients in a vast array of cosmetic products, contributing not only to their texture and consistency but also to their sensory appeal and overall effectiveness. With the growing consumer preference for natural, cruelty-free, and sustainable products, there is an imperative need for groundbreaking solutions that can meet these evolving demands while still providing the desired sensory and performance attributes. In addition, there is a need to move away from micro-plastics and polymers that have been used throughout the beauty and cosmetics industries. The introduction of plant-based fat ingredients and delivery systems, with its capability to mirror the sensory properties of traditional fats, is a significant step forward in aligning cosmetics with the values of conscious consumers and addressing the environmental concerns prevalent in the beauty industry.

### SUMMARY OF THE INVENTION

In its broadest sense, the present invention relates to modified fungal pellets and their uses. In general, in a first aspect, the subject matter features a composition comprising a fungal pellet, fats and/or oils. The fats and/or oils, in an embodiment, are exogenous fats and/or oils that are not derived from the fungus that generates the fungal pellets. The fungal pellet may contain fats and/or oils in the fungal pellet or more particularly, within mycelium hyphae. The composition may further comprise the incorporation of an emulsion of fat contained in the mycelium hyphae of the fungal pellet. The fungal pellets may contain additional fats and/or oils absorbed into the interstitial space of the fungal pellet. The fungal pellets may contain an emulsion, and/or an oleogel, and/or a hydrocolloid containing fats and/or oils, wherein one or more of the emulsions, oleogels, or hydrocolloids are absorbed into the interstitial space of the fungal pellet.

In some aspects, the composition may further comprise additional fats and/or oils contained within the mycelium having formed an additional layer, wherein the additional layer of the pellet is exterior to the first layer. The composition may further comprise additional fats and/or oils contained within the mycelium, wherein the first layer contains fats and/or oils, a middle layer with fats and/or oils, and an outer mycelium layer with fats and/or oils. The fungal pellets may contain one or more layers with fats and/or oils and where additional fats and/or oils are absorbed into the interstitial space of the fungal pellet. The fungal pellets may contain an emulsion, and/or an oleogel, and/or a hydrocolloid containing fats and/or oils, wherein one or more of the emulsions, oleogels, or hydrocolloids are absorbed into the interstitial space of the fungal pellet. In other aspects, the fungal pellet may be encapsulated with a shell (which can also be referred to as a coating) to contain and control the release of the fats and/or oils, and/or ingredients. The layers of fats and/or oils also contributes to the stability and changes the release kinetics of constituent parts of the fungal pellet. Interior layers of the fungal pellet and the fats and/or oils that may be contained therein are more protected from oxidation than the outer layers.

The fungal pellets may contain fat and/or oils, nutrients, and emulsions, derived from agricultural processes and products, including raw agricultural goods, agricultural waste streams and modified agricultural goods. The size of the pellets may be between 10 microns to 100 millimeters. 50 microns to as large as 20 millimeters. The fungal pellet may comprise from about 10 wt % to about 90 wt % fat, based on the total dry weight of the fungal pellet. The fat may be a high melting point fat having a melting point of 36° C. to 110° C. or a low melting point fat having a melting point of 1° C. to 36° C.

Turning to the physical parameters of the fungal pellets, these pellets can exhibit a range of sizes from as small as 50 microns to as large as 20 millimeters. Alternatively, the fungal pellet size may range from 50-200 microns, or from 100-300 microns, or from 200-500 microns, or from 0.5-1 millimeters, or from 0.5-3 millimeters, or from 1-4 millimeters, or from 2-5 millimeters, or from 3-6 millimeters, or from 4-8 millimeters, or from 5-10 millimeters, or from 7-12 millimeters, or from 8-14 millimeters, or from 10-16 millimeters, or from 12-20 millimeters.

The fungal pellet may comprise from about 10 wt % to about 90 wt % fat (and the fat may be endogenous or exogenous), based on the total dry weight of the fungal pellet. Alternatively, the fat content may range from 10-20 wt %, or from 10-30 wt %, or from 10-40 wt %, or from 20-50 wt %, or from 30-60 wt %, or from 30-70 wt %, or from 40-80 wt %, or from 50-90 wt %.

The fat may be characterized by melting point ranges, including high melting point fats having a melting point from 36° C. to 110° C. Alternatively, the melting point range may be from 36-50° C., or from 50-70° C., or from 70-90° C., or from 90-110° C. Low melting point fats may have a melting point range of from 1° C. to 36° C. Alternatively, the melting point ranges may be from 1-10° C., or from 10-20° C., or from 20-30° C., or from 30-36° C.

In further aspects, the fats and/or oils may comprise plant-based fats, animal fats, cell cultured animal fats, synthetic fats, triglycerides, triacylglycerols, caproic acid, caprylic acid, 12-hydroxystearic acid, 9-hydroxystearic acid, 10-hydroxystearic acid, castor oil, algal oil, microbial oil, fish oil, palm oil, palm kernel oil, rapeseed oil, sunflower oil, coconut oil, canola oil, soybean oil, flaxseed oil, wheat germ oil, corn oil, rice oil, olive oil, cottonseed oil, safflower oil, sesame oil, argan oil, walnut oil, almond oil, babassu oil, shea butter, shea kernel oil, mango butter, cocoa butter, borage oil, black currant oil, sea-buckthorn oil, macadamia oil, saw palmetto oil, rice bran oil, peanut oil, linolenic acid, gamma linolenic acid, alpha-linolenic acid, eicosapentaenoic acid, docosahexaenoic acid, stearidonic acid, eicosatetraenoic acid and combinations thereof. In some aspects, the emulsions may be derived from emulsifiers including proteins, celluloses, single glycerides, double glycerides, lactylated glycerides, acylated glycerides, alkoxylated glycerides, glyceride esters connected with diacetyl tartaric acid, phospholipids, lecithin, egg-derived lecithin, soy-derived lecithin, starch modified with succinic acid, modified corn starch, gum Arabic, gum Arabic derivatives with succinic acid, saponins from Quillaya, fatty acid salts of magnesium, fatty acid salts of potassium, fatty acid salts of calcium, polysorbates, fatty acid lactylates from alkali metals, fatty acid lactylates from alkaline earth metals, esters derived from sugars, sodium phosphates, sodium dodecyl sulfate, cetyl Alcohol, stearyl alcohol, cetearyl alcohol, myristyl alcohol, behenyl alcohol, oleyl alcohol, lauryl alcohol, isostearyl alcohol, lanolin alcohol, arachidyl alcohol, isocetyl alcohol, beeswax, dyes, and Guar Gum, or any combination thereof.

In other aspects, the oleogel material that is added may comprise a material selected from the group consisting of pullulan, alginate, cross linked alginate, sodium alginate, propylene glycol alginate, pectin, amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, chitosan, and combinations thereof.

In further aspects, the hydrocolloid material that is added may comprise a material selected from the group consisting of pullulan, alginate, cross linked alginate, sodium alginate, propylene glycol alginate, pectin amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, heparin, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, chitosan, and a combination thereof.

In some aspects, the shell may comprise wax, alginate, chitin, protein polymer gel, and combinations thereof.

The fungus may be selected from the group consisting of Aspergillus sp., Penicillium sp., Agaricus sp., Amanita sp., Armillaria sp., Auricularia sp., Boletus sp., Bovista sp., Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp., and a combination thereof.

Materials that can be added may include but are not limited to additives that provide flavors, colors, and/or scents, along with other materials that are nutrients, minerals, vitamins, salts, as well as combinations thereof. In other aspects, a food product may contain the fungal pellet and fat composition. The food product may be a burger patty, sausage, plant-based meat substitute, dairy alternative, baked good, snack, confectionery, dessert, processed food, gluten-free product, non-dairy whipped topping or other food reliant on fats and/or oils for enhancing taste, texture of sensory properties. A cosmetic product may contain the fungal pellet fat and/or oil composition. The cosmetic product may be a cream, moisturizer, soap and detergents, personal care products, cleanser, sunscreen, serum, lip balm, body oil, hair oil, makeup, perfume, shaving cream, shaving gel, anti-aging cream, hand cream, body butter, massage oil, or baby care product.

In further aspects, a method of encapsulating fats and/or oils in the mycelium hyphae of pellets may comprise growing the pellets in a media containing one or more fat and/or oils including plant-based fat and/or oils, animal fat and/or oils, cell cultured animal fat and/or oils, or synthetic fats and/or oils. The method may further comprise creating a core shell structure using different fats and/or oils and flavors during the growth of the mycelium hyphae. The method may further comprise drying or freeze drying the mycelium fat pellets in a post processing step to increase the amount of fat that can be absorbed in the interstitial space of the pellets. The method may further comprise creating a multi-layer fat and tuning the various layers in the fungal pellet to mimic the texture, taste, color, and/or appearance of animal derived fat. The method may further comprise integrating fat flavors during the growth of mycelium to capture the taste, texture, and mouthfeel of traditional fats and/or oils and other components, such as dyes, if they are present.

The method may further comprise using oleogel or oil and gums to recreate familiar tastes and textures compared to animal fats and/or oils. The method may further comprise using mycelium pellets during encapsulation and post processing to enable an optimized surface to volume ratio absorption of fats and/or oils. The method may further comprise incorporating the mycelium encapsulated fats and/or oils into different stages of mycelium pellet growth to achieve a differential core-layer structure. The method may further comprise loading the mycelium encapsulated fats and/or oils with additional fats and/or oils during post processing through water/oil exchange or drying followed by saturation with fats.

The method may further comprise using the mycelium encapsulated fats and/or oils to improve the taste, texture, and flavor of alternative meat products, improve the appearance of alternative meat products, extend the release of fats during the cooking process, and/or improve the delivery of flavors incorporated into fats. The method may further comprise using the mycelium encapsulated fats and/or oils to provide the visual implied texture, retention of fat during the cooking process, and/or delivery of flavor during the cooking process. The method may further comprise using the mycelium encapsulated fats and/or oils to improve the function, controlled release and other properties of cosmetics.

### BRIEF DESCRIPTION OF SEVERAL VIEWS OF THE DRAWINGS

FIG. 1A illustrates three regions of a fungal pellet as well as a cross section of a layered fungal pellet.

FIG. 1B is four photographs of the layered pellet as both a whole fungal pellet and fungal pellet cross section.

FIG. 2A is a photograph of a plant-based burger with the fat encapsulated fungal pellet incorporated into the material and pressed into a patty.

FIG. 2B is a photograph of a cooked plant-based pepperoni with the fat encapsulated fungal pellet incorporated into the material.

FIG. 3 shows a graph of oil released over time during the cooking process. The patty is grilled at 325° F. for eight minutes, turned at 4 minutes and plotted at one minute intervals.

FIG. 4A shows a graph of viscosity as a function of shear rate for an oil in water fungal pellet emulsion.

FIG. 4B shows a graph of shear stress as a function or shear rate for the emulsion.

FIG. 4C is a photograph of the emulsion spread on a tray at room temperature.

FIG. 4D is a photograph of the emulsion spread after 12 hours at room temperature.

FIG. 5A shows a graph illustrating the toughness of fungal pellets grown at 3 media strength concentrations.

FIG. 5B shows a graph illustrating the % resilience of fungal pellets grown at 3 media strength concentrations.

FIG. 6 shows an overlay of FID gas chromatographs for a fragrance compound and peaks found in the fungal pellet samples.

FIG. 7A shows a SEM image a dehydrated fungal pellet.

FIG. 7B shows a light microscope image at 60× magnification of oil droplets within the hyphae of a dehydrated fungal pellet.

### DETAILED DESCRIPTION

### Definitions

The term “encapsulation” as used herein refers to the formation of a complete or partial barrier around a particle or an object for specifically controlling the movement of substances into or out of encapsulated particle or object.

The term “shell” or “coating” as used herein refers to a layer of the composition created on the exterior of an object, such as on the exterior of a fungal pellet of the invention. The layer need not have a uniform thickness or be completely homogenous in composition. The shell or coating need not cover the entire object to which it is applied. In some embodiments, the film or coating can substantially coat the object. In such embodiments, the film or coating can cover about 10%, 20%, 30%, 40%, 50%, 60%, 70%, 80%, or about 90% of the surface area of the object. In other embodiments, the film or coating can completely coat the object—that is it can cover about 100% of the object. In some embodiments, the film or coating can have a thickness that varies by about 10%, 20%, 30%, 40%, 50%, 60%, 70%, 80%, or about 90% over the object.

The term “interstitial space” as used herein refers to the open spaces or gaps within the network of fine, thread-like filaments that make up the mycelium structure within the pellet and has biologic function for the fungi by delivering of nutrients, growth and biologic signaling.

In an embodiment, the present disclosure relates to a composition comprising a fungal pellet, which is a structure formed by the growth of mycelium, a part of a fungus. The mycelium forms a network of filamentous structures known as hyphae, which collectively make up the bulk of the fungal pellet.

In some cases, the fungal pellet may contain fats and/or oils within the mycelium hyphae. This incorporation can occur during the growth of the mycelium, where the fats and/or oils are absorbed by the hyphae and become an integral part of the fungal pellet structure.

Fats and/or Oils in Interstitial Space

In some cases, the fungal pellet can be dried and saturated with fats and/or oils, which surprisingly enables much larger amounts of fats and/or oils to be encapsulated and loaded within the fungal pellet.

In some cases, the fungal pellet may contain additional layers of fats and/or oils and/or dyes and/or biological molecules. These additional layers can be formed within the mycelium, creating a layered structure within the fungal pellet. The layers can contain different types of fats and/or oils, providing a range of sensory properties and nutritional profiles. The additional layers of fats and/or oils can be exterior to the first layer of the pellet, forming a layered structure that can be tailored to specific applications.

Furthermore, the fungal pellets may contain emulsions, oleogels, or hydrocolloids containing fats and/or oils. An emulsion is a mixture of two or more liquids that are normally immiscible, such as oil and water. In the context of the fungal pellet, the emulsion can be formed by the fats and/or oils and a suitable emulsifier, which helps to stabilize the emulsion. An oleogel is a semi-solid gel formed by the combination of an oil and a gelling agent, which can provide a range of textures and sensory properties. A hydrocolloid is a substance that forms a gel in contact with water, and can be used to stabilize emulsions and improve the texture of the fungal pellet. The incorporation of these components within the fungal pellet can enhance the sensory properties and nutritional profile of the pellet, making it a versatile ingredient for a range of applications.

Delving deeper into the composition of the fungal pellet, it is primarily composed of mycelium hyphae, which are the thread-like components of the fungus that form a network-like structure. Within these mycelium hyphae, fats and/or oils are contained. These fats and/or oils can be derived from a variety of sources, including but not limited to plant-based fats, animal fats, cell cultured animal fats, and synthetic animal fats. The incorporation of these fats and/or oils within the mycelium hyphae is a distinctive feature of the fungal pellet, contributing to its nutritional profile and sensory properties.

In some instances, the fungal pellet may contain additional fats and/or oils. These additional fats and/or oils can be located within the mycelium hyphae, further enhancing the fat content of the fungal pellet. Alternatively, these additional fats and/or oils can be absorbed into the interstitial space of the pellet. The interstitial space refers to the spaces between the mycelium hyphae, providing additional capacity for the absorption of fats and/or oils. This absorption can occur during the growth of the mycelium, where the fats and/or oils are absorbed by the hyphae and become an integral part of the fungal pellet structure.

Moreover, the fungal pellet may contain an emulsion of fat, an oleogel, and/or a hydrocolloid containing fats and/or oils. An emulsion of fat refers to a mixture of fat and another liquid, typically water, that are normally immiscible but are made to form a stable mixture with the help of an emulsifier. An oleogel is a semi-solid gel formed by the combination of an oil and a gelling agent, providing a range of textures and sensory properties. A hydrocolloid is a substance that forms a gel in contact with water, and can be used to stabilize emulsions and improve the texture of the fungal pellet. The incorporation of these components within the fungal pellet can enhance the sensory properties and nutritional profile of the pellet, making it a versatile ingredient for a range of applications.

In some instances, the fungal pellet may exhibit a layered structure, with additional layers of fats and/or oils contained within the mycelium. These layers can be formed during the growth of the mycelium, where the fats and/or oils are absorbed by the hyphae and become an integral part of the fungal pellet structure. The layers can contain different types of fats and/or oils, providing a range of sensory properties and nutritional profiles. For example, one layer may contain plant-based fats, while another layer may contain animal fats, cell cultured animal fats, or synthetic animal fats. This layered structure can be tailored to specific applications, offering flexibility in the design of the fungal pellet.

Furthermore, these additional layers of fats and/or oils can be exterior to the first layer of the pellet. This means that the fats and/or oils are not just contained within the mycelium hyphae but can also form an outer layer around the pellet. This outer layer can serve multiple functions. For instance, it can provide a barrier that protects the inner contents of the pellet, or it can serve as a reservoir that releases fats and/or oils during cooking or other processing steps. This exterior layer of fats and/or oils can enhance the sensory properties of the fungal pellet, contributing to its taste, texture, and mouthfeel. In one variation, omega-3 fats can be added to the fats and/or oils, which provides the properties that omega-3 fats are known to provide.

Turning to the physical parameters of the fungal pellets, these pellets can exhibit a range of sizes, from as small as 50 microns to as large as 20 millimeters. Alternatively, the fungal pellet size may range from 50-300 microns, or from 50-500 microns, or from 0.5-2 millimeters, or from 0.5-4 millimeters, or from 0.5-6 millimeters, or from 2-4 millimeters, or from 2-7 millimeters, or from 2-10 millimeters, or from 5-10 millimeters, or from 5-15 millimeters, or from 5-20 millimeter, or from 8-12 millimeter, or from 8-16 millimeter, or from 8-20 millimeter. This size range provides flexibility in the design of the fungal pellet, allowing it to be tailored to specific applications. For instance, smaller pellets may be suitable for applications where a fine texture is desired, while larger pellets may be used in applications where a chunkier texture is preferred. Also, where the size can play a critical role is in delivery, where a small size may deliver specific nutrients in cosmetic skin applications, while other larger pellets may be used where delivery of fragrance and flavor release.

Furthermore, the fungal pellets can contain a substantial amount of fat, with the weight percentage of fat within the pellet potentially ranging from about 10 wt % to about 90 wt %. Alternatively, the relative amount of fat within the pellet may range from 10-20 wt %, or from 10-30 wt %, or from 10-40 wt %, or from 10-50 wt %, or from 20-30 wt %, or from 20-40 wt %, or from 20-50 wt %, or from 30-40 wt %, or from 30-50 wt %, or from 30-60 wt %, or from 30-70 wt %, or from 30-80 wt %, or from 40-50 wt %, or from 40-60 wt %, or from 40-70 wt %. This high fat content contributes to the nutritional profile of the fungal pellet, providing a source of energy and aiding in the absorption of fat-soluble vitamins. The high fat content also enhances the sensory properties of the pellet, contributing to its taste, texture, and mouthfeel. Surprisingly, when dehydrated and then saturated in fats and/or oils the fungal pellet can be loaded fats and/or oils totaling 10% to more to over 200% of its dry weight.

Moreover, the fats and/or oils contained within the fungal pellet can exhibit a range of melting points, from as low as 1° C. to as high as 110° C. In a variation, the melting point range may be low temperature melting points, for example melting points that are between about 1° C. to 36° C., or higher melting points from 36-110° C., or alternatively, melting points that are in a range of 50-110° C. or 60-110° C. or 70-110° C. or 608-110° C. In its broadest sense, this wide melting point range allows for the tuning of the sensory properties of the fungal pellet. For example, fats with a low melting point can provide a melt-in-your-mouth sensation, while fats with a high melting point can contribute to the firmness and chewiness of the pellet. The melting point of the fats and/or oils can also influence the cooking properties of the pellet, affecting how it behaves when heated and how it releases its fats and/or oils during cooking.

Delving into the types of fats and/or oils that can be used in the composition, a wide range of options are available. These include plant-based fats, animal fats, cell cultured animal fats, and synthetic animal fats. Plant-based fats can be derived from a variety of sources, such as soybean, canola, sunflower, corn, palm, olive, and coconut oil. These vegetable oils are derived from plant sources and can provide a range of flavors and textures, as well as beneficial nutrients such as unsaturated fats and vitamins.

Animal fats, on the other hand, can be derived from various animal sources, including but not limited to beef, pork, chicken, and fish. These animal fats can provide a rich, savory flavor and a creamy texture, enhancing the sensory properties of the fungal pellet. Cell cultured animal fats are a novel type of fat that is produced by culturing animal cells in a lab, providing a sustainable and ethical alternative to traditional animal fats. Synthetic animal fats are another innovative type of fat that is chemically synthesized to mimic the properties of animal fats, offering another alternative for those seeking to avoid animal products.

Triglycerides and triacylglycerols are specific types of fats that are composed of three fatty acids attached to a glycerol backbone. These types of fats can be derived from both plant and animal sources, and they provide a rich source of energy. They also contribute to the texture and mouthfeel of the fungal pellet, enhancing its sensory properties.

Furthermore, a variety of vegetable oils can be used in the composition. These can include soybean oil, canola oil, sunflower oil, corn oil, palm oil, olive oil, and coconut oil, among others. These vegetable oils are derived from plant sources and can provide a range of flavors and textures, as well as beneficial nutrients such as unsaturated fats and vitamins. The use of these diverse types of fats and/or oils provides flexibility in tailoring the sensory properties and nutritional profile of the fungal pellet, making it a versatile ingredient for a range of applications.

Delving into the components of the fat emulsions within the fungal pellet, a variety of emulsifiers can be utilized. Emulsifiers are substances that help stabilize emulsions, preventing the immiscible liquids from separating. In the context of the fungal pellet, the emulsifiers can help stabilize the fat emulsion, enhancing the texture and sensory properties of the pellet.

One type of emulsifier that can be used is proteins. Proteins are large, complex molecules that in some instances can act as emulsifiers due to their amphiphilic nature, meaning they have both hydrophilic (water-loving) and hydrophobic (water-repelling) parts. This allows them to interact with both the fat and water components of the emulsion, helping to stabilize it. Celluloses can also be used as emulsifiers. Celluloses are polysaccharides, or complex carbohydrates, that are composed of glucose units. They can form a network structure that can trap and stabilize the fat droplets within the emulsion, preventing them from coalescing and separating from the water phase.

Single glycerides, double glycerides, lactylated glycerides, acylated glycerides, and alkoxylated glycerides are other types of emulsifiers that can be used. These are all derivatives of glycerol, a simple molecule that forms the backbone of fats and oils. They can interact with both the fat and water components of the emulsion, helping to stabilize it.

Glyceride esters connected with diacetyl tartaric acid can also be used as emulsifiers. These are complex molecules that have both hydrophilic and hydrophobic parts, allowing them to interact with both the fat and water components of the emulsion and stabilize it.

Phospholipids, such as lecithin, can also be used as emulsifiers. Phospholipids are a type of lipid that is a major component of cell membranes. They have a hydrophilic “head” and two hydrophobic “tails”, allowing them to interact with both the fat and water components of the emulsion and stabilize it. Lecithin, which can be derived from sources such as eggs (egg-derived lecithin) or soy (soy-derived lecithin), is a common type of phospholipid used as an emulsifier.

In summary, a variety of emulsifiers can be used to stabilize the fat emulsions within the fungal pellet, enhancing its texture and sensory properties. These emulsifiers can interact with both the fat and water components of the emulsion, preventing them from separating and ensuring the stability of the pellet.

Turning to the materials that can be used to form the oleogels and hydrocolloids within the fungal pellet, a variety of options are available. One such material is pullulan, a polysaccharide polymer that is, for example, produced from starch by the fungus Aureobasidium pullulans. Pullulan has the ability to form a gel in the presence of water, making it a suitable material for forming oleogels and hydrocolloids.

Alginate, a polysaccharide derived from brown seaweed, can also be used to make oleogels. Alginate has the ability to form a gel in the presence of divalent cations, such as calcium, making it a suitable material for forming oleogels and hydrocolloids. In some cases, cross linked alginate, sodium alginate, or propylene glycol alginate may be used, each offering different properties and characteristics that can be tailored to specific applications.

Pectin, a polysaccharide found in the cell walls of plants, can also be used to form oleogels and hydrocolloids. Pectin has the ability to form a gel in the presence of sugar and acid, making it a suitable material for these applications. Specific types of pectin, such as pectin amylopectin and methoxyl pectin, may be used depending on the desired properties of the oleogel or hydrocolloid.

Inulin, a polysaccharide found in many plants, can also be used. Inulin has the ability to form a gel in the presence of water, making it a suitable material for forming oleogels and hydrocolloids.

Carrageenan, a polysaccharide extracted from red edible seaweeds, is another material that can be used. Carrageenan has the ability to form a gel in the presence of potassium ions, making it a suitable material for these applications.

Cellulose gum, also known as carboxymethyl cellulose, can also be used to form oleogels and hydrocolloids. Cellulose gum is a derivative of cellulose, the main component of plant cell walls, and has the ability to form a gel in the presence of water. Other types of gums, such as xanthan gum, locust bean gum, gellan gum, guar gum, tara gum, and konjac gum, can also be used. These gums are polysaccharides that have the ability to form a gel in the presence of water, making them suitable materials for forming oleogels and hydrocolloids.

In summary, a variety of materials can be used to form the oleogels and hydrocolloids within the fungal pellet. These materials can be selected based on their ability to form a gel in the presence of water or other substances, their source, and their impact on the sensory properties and nutritional profile of the fungal pellet.

Another aspect of the fungal pellet composition involves the potential use of a shell material to generate a shell and encapsulate the pellet. This shell can serve multiple functions, such as protecting the inner contents of the pellet, controlling the release of fats and/or oils during cooking or other processing steps, and enhancing the sensory properties of the pellet.

One potential material for the shell is wax. Wax is a broad class of organic compounds that are lipophilic, malleable solids near ambient temperatures. They include higher alkanes and lipids, typically with melting points above about 40° C. (104° F.), melting to give low viscosity liquids. Waxes are insoluble in water but soluble in organic, nonpolar solvents. Wax can provide a robust and water-resistant shell that can protect the inner contents of the pellet and control the release of fats and/or oils.

Alginate is another potential material for the shell. Alginate is a polysaccharide derived from brown seaweed that has the ability to form a gel in the presence of divalent cations, such as calcium. When used as a shell material, alginate can form a semi-permeable membrane around the pellet, allowing for controlled release of fats and/or oils.

Chitin, a long-chain polymer of N-acetylglucosamine, is another potential shell material. Chitin is a primary component of cell walls in fungi, the exoskeletons of arthropods, such as crustaceans and insects, and the scales of fish and lissamphibians. It provides a robust and biodegradable shell that can protect the inner contents of the pellet and control the release of fats and/or oils.

Protein polymer gel is another potential shell material. Protein polymer gels are formed by the cross-linking of protein molecules, creating a three-dimensional network that can encapsulate the pellet. The properties of the protein polymer gel can be tailored by adjusting the degree of cross-linking, allowing for control over the shell's strength, permeability, and other properties. In some cases, a combination of these materials may be used to form the shell. For instance, a shell composed of a wax outer layer and an alginate inner layer may provide both robust protection and controlled release of fats and/or oils. The choice of shell material or combination of materials can be tailored to the specific requirements of the application, providing flexibility in the design of the fungal pellet. In one variation, alginate used alone or combined with the other materials disclosed herein, can be used as a shell material.

Turning to the fungal strains that can be used to form the mycelium hyphae, a variety of options are available. These include Aspergillus sp., Penicillium sp., Agaricus sp., Amanita sp., Armillaria sp., Auricularia sp., Boletus sp., Bovista sp., Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp.

These fungal strains are selected based on their ability to form mycelium hyphae, which occurs in most filamentous fungi. The mycelium hyphae is a thread-like components of the fungus that form a network-like structure. The mycelium hyphae serve as the primary structure of the fungal pellet, providing a scaffold for the incorporation of fats and/or oils. The choice of fungal strain can influence the growth characteristics of the mycelium, the structure of the fungal pellet, and the sensory properties of the pellet. For instance, some fungal strains may produce mycelium hyphae that are particularly suited to absorbing fats and/or oils, while others may produce hyphae that form a particularly robust or porous pellet structure.

Furthermore, the choice of fungal strain can also influence the nutritional profile of the fungal pellet. For instance, some fungal strains may produce mycelium that is rich in protein, fiber, or other nutrients, enhancing the nutritional value of the pellet.

The fungal strain can also influence the taste, texture, and mouthfeel of the pellet, contributing to its sensory properties. Therefore, the choice of fungal strain is a pivotal aspect of the design of the fungal pellet, providing flexibility in tailoring the pellet to specific applications.

Further enhancing the versatility of the fungal pellet composition, a variety of additives can be incorporated into the pellet. These additives can serve multiple functions, such as enhancing the sensory properties of the pellet, improving its nutritional profile, or providing functional benefits. Flavors are one type of additive that can be included in the composition. These can range from natural flavors derived from fruits, vegetables, spices, and other food sources, to artificial flavors that mimic the taste of these natural sources. The choice of flavor can be tailored to the specific application of the fungal pellet, allowing for a wide range of taste profiles. For instance, savory flavors may be used for applications in meat substitutes, while sweet flavors may be used for applications in baked goods or desserts.

Colors can also be added to the fungal pellet composition. These can be natural colors derived from fruits, vegetables, and other food sources, or artificial colors that mimic the appearance of these natural sources. The addition of colors can enhance the visual appeal of the fungal pellet, making it more attractive to consumers. For instance, red or brown colors may be used to mimic the appearance of meat, while green or yellow colors may be used to indicate the presence of vegetables or fruits.

Scents can be another type of additive included in the composition. These can be derived from natural sources, such as herbs, spices, and flowers, or from artificial sources that mimic these natural scents. The addition of scents can enhance the sensory experience of the fungal pellet, contributing to its overall appeal. For instance, a smoky scent may be used for applications in barbecue-flavored products, while a sweet scent may be used for applications in baked goods or desserts.

Nutrients, minerals, and vitamins can also be added to the fungal pellet composition. These can enhance the nutritional profile of the pellet, making it a healthier choice for consumers. For instance, vitamins A, C, and E can be added for their antioxidant properties, while B vitamins can be added for their role in energy metabolism. Minerals such as calcium, iron, and zinc can be added for their role in various bodily functions. The addition of these nutrients, minerals, and vitamins can make the fungal pellet a source of valuable nutrition, in addition to its role as a source of fats and/or oils.

Salts can also be included in the composition. These can enhance the taste of the fungal pellet, bringing out the flavors of the other ingredients. The type of salt used can be tailored to the specific application of the pellet. For instance, sea salt or kosher salt may be used for their coarse texture and robust flavor, while table salt or iodized salt may be used for their fine texture and mild flavor.

In some cases, a combination of these additives may be used. For instance, a combination of flavors, colors, and scents may be used to create a multi-sensory experience, while a combination of nutrients, minerals, and vitamins may be used to enhance the nutritional profile of the pellet. The choice of additives can be tailored to the specific requirements of the application, providing flexibility in the design of the fungal pellet.

The fungal pellet fat composition disclosed herein can be utilized in a variety of food products. For instance, it can be incorporated into burger patties or sausages, where it can contribute to the juiciness and flavor of the product. The composition can also be used in plant-based meat substitutes, where it can mimic the taste and texture of animal fat, enhancing the sensory appeal of these products. Dairy alternatives, such as plant-based cheeses, yogurts, or milks, can also benefit from the inclusion of the fungal pellet fat composition, where it can provide a creamy texture and rich flavor. Furthermore, the structure of the fungal pellet has a critical role in retaining oils, dyes, flavors, and nutrients in these products to further enhance their taste, texture, stability, and use.

Furthermore, the composition can be used in baked goods, such as breads, pastries, or cookies. Here, the fungal pellet fat composition can contribute to the moistness and flavor of the baked goods, enhancing their sensory properties. Snacks, such as chips or popcorn, can also benefit from the inclusion of the composition, where it can provide a satisfying crunch and rich flavor. Confections, such as chocolates or candies, can also incorporate the composition, where it can contribute to the smooth texture and indulgent flavor of these treats.

Desserts, such as ice cream or pudding, can also utilize the fungal pellet fat composition. Here, the composition can provide a creamy texture and rich flavor, enhancing the sensory appeal of these desserts. Processed foods, such as ready meals or canned foods, can also incorporate the composition, where it can contribute to the flavor and mouthfeel of these products. Gluten-free products, such as gluten-free breads or pastries, can also benefit from the inclusion of the composition, where it can provide a moist texture and rich flavor. Non-dairy whipped toppings can also utilize the composition, where it can provide a light, fluffy texture and rich flavor.

Moreover, the fungal pellet fat composition can also be utilized in cosmetic products. For instance, the fungal pellet(s) can be incorporated into creams or moisturizers, where it/they can provide a smooth, luxurious texture and hydrating properties. Soaps and detergents can also benefit from the inclusion of the composition, wherein it can provide a rich lather and moisturizing properties. Personal care products, such as body washes or shampoos, can also incorporate the composition, where it can provide a creamy texture and moisturizing properties.

Cleansers, such as facial cleansers or body scrubs, can also utilize the fungal pellet fat composition. Here, the composition can provide a smooth, creamy texture and moisturizing properties, enhancing the sensory appeal of these products. Sunscreens can also incorporate the composition, where it can provide a smooth, non-greasy texture and moisturizing properties. Serums, such as facial serums or hair serums, can also benefit from the inclusion of the composition, where it can provide a silky texture and hydrating properties.

Lip balms can also utilize the fungal pellet fat composition, where it can provide a smooth, moisturizing texture and protect the lips from dryness. Body oils or hair oils can also incorporate the composition, where it can provide a rich, nourishing texture and moisturizing properties. Makeup products, such as foundations or lipsticks, can also benefit from the inclusion of the composition, where it can provide a smooth, creamy texture and hydrating properties. Perfumes can also utilize the composition, where it can provide a smooth, non-greasy texture and enhance the longevity of the fragrance.

Shaving creams or shaving gels can also incorporate the fungal pellet fat composition, where it can provide a rich lather and moisturizing properties, enhancing the comfort of the shave. Anti-aging creams can also benefit from the inclusion of the composition, where it can provide a rich, nourishing texture and hydrating properties, helping to reduce the appearance of fine lines and wrinkles. Hand creams or body butters can also utilize the composition, where it can provide a rich, moisturizing texture and hydrating properties. Massage oils can also incorporate the composition, where it can provide a smooth, non-greasy texture and moisturizing properties.

Baby care products, such as baby lotions or baby oils, can also benefit from the inclusion of the composition, where it can provide a gentle, moisturizing texture and hydrating properties.

Turning to the method of encapsulating fats and/or oils in the mycelium hyphae of pellets, this process involves growing the pellets in a media containing one or more types of fats and/or oils. The media can be a liquid or solid substrate that provides the nutrients and conditions for the growth of the mycelium. The exogenous fats and/or oils can be added to the media, where they are absorbed by the growing mycelium hyphae and become an integral part of the fungal pellet structure.

In some cases, the fats and/or oils in the media can be derived from a natural plant source. These plant-based fats and/or oils can provide a range of flavors and textures, as well as beneficial nutrients such as unsaturated fats and vitamins. The media can also contain stabilizing proteins and carbohydrates, including soluble and insoluble fibers. These components can help to stabilize the fat emulsion within the mycelium hyphae, enhancing the texture and sensory properties of the fungal pellet.

During the growth of the mycelium hyphae, a core shell structure can be created using different fats and/or oils and flavors. The core shell structure refers to a structure where the core of the pellet contains one type of fat and/or oil, while the shell or outer layer of the pellet contains a different type of fat and/or oil. This structure can be created by adding the different fats and/or oils and flavors to the media at different stages of the mycelium growth. The core shell structure can enhance the sensory properties of the fungal pellet, providing a range of flavors and textures that can be tailored to specific applications. Although this process has been described with one layer, it should be understood that two or more differential layers can be added, such as three layers, or four layers, or five layers, or more.

In a post processing step, the mycelium fat pellets can be dried or freeze dried to increase the amount of fat that can be absorbed in the interstitial space of the pellets. The interstitial space refers to the spaces between the mycelium hyphae, providing additional capacity for the absorption of fats and/or oils. The drying or freeze drying process can remove water from the pellets, creating more space for the absorption of fats and/or oils. This process can enhance the fat content of the fungal pellet, contributing to its nutritional profile and sensory properties. It should be understood that other compounds as described herein can be added that further contribute to the nutritional profile.

Furthermore, a multi-layer fat can be created and the way the fat interacts can be tuned to mimic the texture, taste, color, and/or appearance of animal derived fat. The multi-layer fat refers to a structure where the fungal pellet contains multiple layers of different types of fats and/or oils. Although the process is described with reference to fats and/or oils, it should be understood that other molecules/compounds can be encapsulated in the manner described here. For example, flavoring molecules, nutrients, vitamins, molecules that provide good mouth feel or smell, or other molecules that are described herein can be added.

This structure can be created by adding the different types of fats and/or oils to the media at different stages of mycelium growth. The interaction of the fats and/or oils can be tuned by adjusting the types and proportions of fats and/or oils used, as well as the conditions of the mycelium growth and post processing steps.

Thus, fungal pellets can be created that have different characteristics depending on what layer of the fungal pellet is referenced. For example, the outermost layer in a fungal pellet may contain one concentration of fats and/or oils (and possibly other compounds) that provides the pellet with a different mouth feel relative to an inner layer that has a different compositional makeup and/or different concentrations. This inner layer, which has the different fats and/or oils and other molecules may provide the fungal pellet with a different taste sensation than the outermost layer. Accordingly, by employing differential layers, one can fine tune the fungal pellet to have any of desired characteristics and those desired characteristics can be placed at any layer of the fungal pellet.

During the growth of mycelium, fat flavors can be integrated to capture the taste, texture, and mouthfeel of traditional fats and/or oils. The fat flavors can be derived from natural sources, such as herbs, spices, fruits, and vegetables, or from artificial sources that mimic these natural flavors. The integration of fat flavors can enhance the sensory appeal of the fungal pellet, making it a versatile ingredient for a range of applications.

Oleogel or oil and gums can also be used to recreate familiar tastes and textures compared to animal fats and/or oils. Thus, in an embodiment, the fungal pellets can be used in any of a plurality of different food compositions. An oleogel is a semi-solid gel formed by the combination of an oil and a gelling agent, providing a range of textures and sensory properties. Gums are polysaccharides that have the ability to form a gel in the presence of water, enhancing the texture of the fungal pellet. The use of oleogel or oil and gums can provide a familiar taste and texture, making the fungal pellet a suitable alternative to animal fats and/or oils in a variety of applications.

Finally, mycelium pellets can be used during encapsulation and post processing to enable an optimized surface to volume ratio absorption of fats and/or oils. The encapsulation process involves surrounding the fats and/or oils with a layer of mycelium, forming a pellet. The post processing steps can involve drying or freeze drying the pellets, creating more space for the absorption of fats and/or oils. The use of mycelium pellets during encapsulation and post processing can enhance the fat content of the fungal pellet, contributing to its nutritional profile and sensory properties.

The present invention further describes a method for creating emulsions using fungal pellets by applying mechanical force through processes such as blending, shearing, homogenization, or other forms of mechanical disruption to partially disrupt the structural integrity of the fungal pellet. In this method, the mechanical disruption targets the interstitial spaces of the fungal pellet while preserving the encapsulation of fats, oils, and compounds within the hyphal structures. This process allows for the incorporation and encapsulation of both water-soluble and oil-soluble compounds, including classes of bioactive compounds such as carotenoids (e.g., beta-carotene, lycopene), polyphenols (e.g., quercetin, resveratrol), and vitamins (e.g., vitamin C, vitamin E). It also allows for the inclusion of flavors, colors, and other sensory components to achieve desired characteristics in the final product. This facilitates the controlled delivery and gradual release of these compounds under specific conditions. The resulting emulsions have been observed to exhibit non-Newtonian shear-thinning behavior, indicating a reduction in viscosity with increased shear rates, which enhances their processability and applicability in various formulations. The disruption of the interstitial space facilitates the interaction of encapsulated compounds with external media, forming a stable emulsion while retaining the encapsulated fats, oils, and active agents within the fungal pellet.

The emulsification process enables the inclusion of various fats and oils that were initially encapsulated within the hyphae during the growth of the fungal pellet. Additionally, other ingredients, such as texturizing agents, flavors, supplementary oils, water-soluble vitamins, antioxidants, colors, or bioactive compounds, can be incorporated during the emulsification phase. This approach allows for the development of emulsions that provide tailored sensory and functional properties. The resulting emulsion demonstrates emergent properties that are enhanced compared to the biomass of the whole pellet before homogenization, due to the synergistic effects of the disrupted pellet structures. The structural elements of the fungal pellet contribute significantly to the stability of the emulsion, offering superior performance over formulations using only purified proteins derived from the mycelium. This enhanced stability is particularly beneficial in food applications such as dairy and dairy alternatives (e.g., cheeses, yogurts, and spreads), dessert products like puddings and mousses, and various culinary bases including creamy sauces and dressings. These emulsions can also be utilized in baked goods and frozen desserts, where they improve moisture retention, mouthfeel, and structural integrity.

In cosmetic formulations, these fungal pellet-based emulsions offer advantages in both stability and controlled release of active ingredients. The encapsulated compounds can include oil-soluble agents such as retinoids or coenzyme Q10 for anti-aging applications, or water-soluble compounds like niacinamide and hyaluronic acid for moisturizing formulations. Additionally, the incorporation of colors and fragrances can enhance the appeal of these emulsions in cosmetic applications. This makes these emulsions suitable for inclusion in a wide range of cosmetic products, including lotions, creams, serums, and sunscreens, where gradual release of actives can enhance product stability and bioavailability. Furthermore, the non-Newtonian shear-thinning properties of these emulsions enable them to provide improved spreading and uniformity during application, while maintaining structural consistency.

Moreover, the enhanced stability provided by the structural elements of the fungal pellet enables these emulsions to be used in multifunctional cosmetic formulations, such as tinted moisturizers or BB creams, where a stable emulsion matrix is required to incorporate active ingredients, colors, and fragrances. This stability also supports formulations like body butters and facial gels, which require a balanced composition of oils, water-based actives, texturizing agents, and sensory additives. The versatility of these emulsions makes them ideal for developing products that offer controlled release and sustained efficacy of encapsulated compounds in various food and cosmetic applications.

By leveraging the structural properties of the fungal pellet, the invention provides a means to produce emulsions with enhanced stability and controlled release of encapsulated compounds. The encapsulation within the hyphal structures ensures that both water-soluble and oil-soluble compounds, including bioactives (and bioactive nutrients), flavors, colors, and dyes that may remain sequestered until specific processing or application conditions trigger their gradual release. This controlled release mechanism, combined with the emergent properties of the emulsion, results in compositions that exhibit desired thermal and mechanical properties, making them suitable for integration into complex food systems and cosmetic products. For example, in food applications, the gradual release of bioactive compounds like carotenoids and polyphenols may enhance nutritional value and antioxidant effects, while in cosmetics, the gradual release of encapsulated compounds such as peptides or natural oils can provide prolonged hydration, anti-aging effects, or enhanced protection from environmental stressors. This broad applicability within food and cosmetic formulations underscores the innovative potential of this fungal pellet-based emulsion technology.

In further aspects, the bioactive nutrients that may be added are selected from the group consisting of beta-carotene, lycopene, tocopherol, sulphoraphane, polyphenols, resveratrol, quercetin, curcumin, astaxanthin, lutein, zeaxanthin, flavonoids, anthocyanins, catechins, epicatechin, isoflavones, glucosinolates, ellagic acid, urolithin A, squalene, allicin, capsaicin, phycocyanin, beta-glucans, saponins, luteolin, genistein, kaempferol, epigallocatechin gallate (EGCG), hesperidin, rutin, cinnamic acid, ferulic acid, chlorogenic acid, oleuropein, beta-sitosterol, gamma-tocotrienol, alpha-lipoic acid, coenzyme Q10, ergothioneine, 1-carnitine, N-acetylcysteine, vitamins including vitamin A, vitamin B complex (including B1, B2, B3, B5, B6, B7, B9, B12), vitamin C, vitamin D, vitamin E, vitamin K, zinc, selenium, copper, magnesium, iron, and a combination thereof.

In further aspects, the colors and dyes that may be added are selected from the group consisting of natural colors, including beet juice, turmeric, spirulina, chlorophyll, paprika extract, annatto, beta-carotene, lycopene, red cabbage extract, blueberry extract, elderberry extract, carrot juice, saffron, matcha powder, hibiscus extract, red radish extract, black carrot extract, grape skin extract, pomegranate extract, spinach powder, cocoa powder, coffee extract, butterfly pea flower extract, caramel color, red algae extract, and red rice yeast. These natural colorants offer a diverse range of hues, including red, pink, purple, yellow, orange, green, and brown, while often providing added health benefits, such as antioxidants, vitamins, or minerals, enhancing the nutritional profile of the food.

Additionally, synthetic or artificial colors may be used to achieve vibrant, stable hues that are challenging to replicate with natural sources. These synthetic colors may include FD&C certified colors such as FD&C Red No. 40, FD&C Blue No. 1, FD&C Yellow No. 5, FD&C Green No. 3, FD&C Yellow No. 6, FD&C Blue No. 2, FD&C Red No. 3, and titanium dioxide, which allow for a broader color spectrum and extended shelf life. Other food-grade dyes in shades such as bright pink, neon green, royal purple, vibrant orange, and rich brown may also be incorporated to meet specific aesthetic goals.

Encapsulated dyes can also be employed to enhance color stability in products exposed to heat, light, or pH changes. The colors, whether natural or synthetic, may be used individually or in combination, enabling precise color customization for the desired visual appeal in various food applications. This could range from pastel tones in baked goods to vivid colors in confectionery, beverages, and processed foods, allowing for layered or gradient effects to enhance visual interest. These colorants align with both functional requirements and consumer expectations for appealing, vibrant, and stable food products.

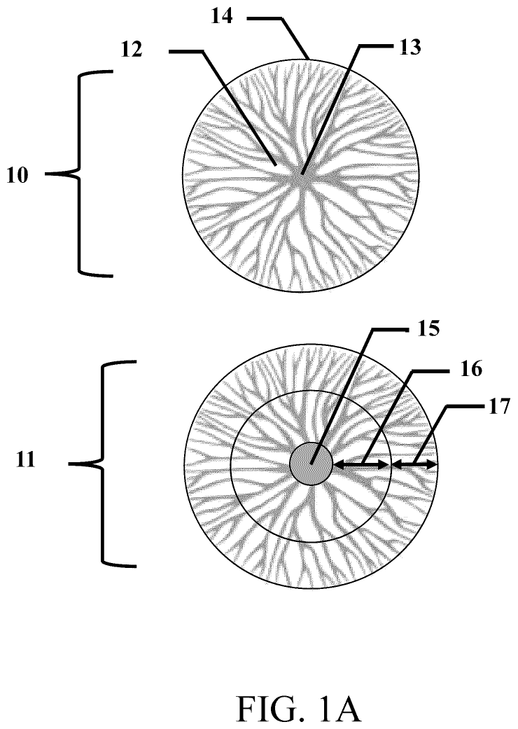

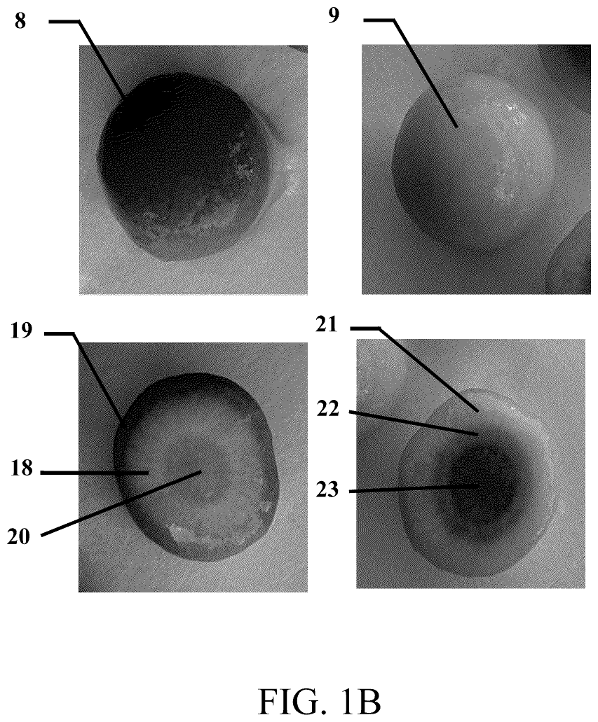

FIG. 1A illustrates cross sections of a fungal pellet (10). The figure illustrates three regions of a fungal pellet regions in which fats and/or oils and/or dyes and/or bioactive compounds can be encapsulated. These regions include interstitial space (12) between hyphae strands (13) intercellular space inside the hyphae strands, and a shell (14) or coating on the exterior of the fungal pellet. Fungal pellet 11 illustrates the surprising property that pellets can comprise 3 (as shown), or alternatively 2, or alternatively 4, or alternatively 5 layers comprising each of the three loading regions having interstitial space, intercellular space, as well as a shell or coating. The core (15) of the fungal pellet is shown with a first layer (16), and an additional outer layer (17).

FIG. 1B shows four photographs illustrating whole fungal pellet, a fungal pellet with a shell or coating (8) a fungal pellet without a shell or coating (9); and cross sectional views of fungal pellets shown in the bottom two photographs. The bottom left hand photograph illustrates a cross sectional view of the shell or coating (19). The outer layer of hyphae (18) is shown encapsulated with one specific compound. The picture also illustrates a cross sectional view of the innermost core (20) of the fungal pellet. The bottom right photograph illustrates an outer layer (21) of a fungal pellet, with a more internal layer (22) comprising a different encapsulated compound relative to the outer layer or the core (23). The cross-sectional photographs demonstrate that a plurality of layers is possible in a fungal pellet. The shell or coating is shown in the bottom left figure as being very thin but it should be understood that depending on the growth conditions (including the time the fungal pellet is grown) the shell may be made to be of any thickness desired. The shell may also include various oils and/or fats and other compounds as disclosed herein.

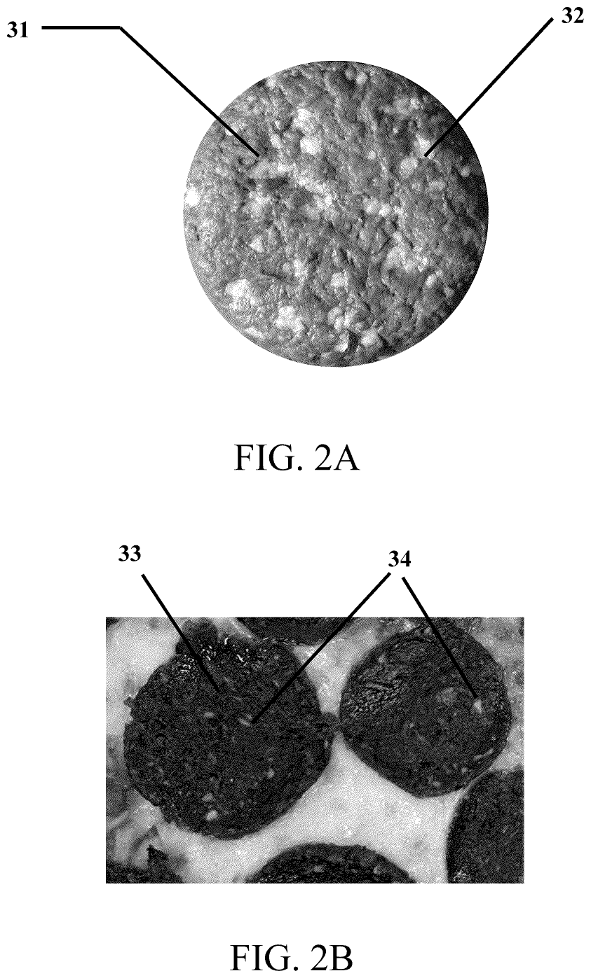

FIG. 2A is a photograph illustrating an application of the fungal pellet with encapsulated plant based oil (high-oleic safflower) applied as a solid particle ingredient within a plant-based burger product. The darker plant-based burger patty base or matrix (31) is shown as are the fungal pellet(s) (32) shown as light spots incorporated into the base as a fat ingredient.

FIG. 2B is a photograph illustrating an application of the fungal pellet with encapsulated plant based oil (high-oleic safflower) applied as a solid particle ingredient within a plant-based pepperoni after the pepperoni's second cook. Pepperoni is cooked during production and also by the consumer. In the pepperoni, the plant-based pepperoni base or matrix (33) is seen as darker regions with the fungal pellet(s) (34) shown as lighter spots incorporated into the base as a fat ingredient. Surprisingly, it was found that the fungal pellet(s) (34) can survive multiple cooks without significant degradation.

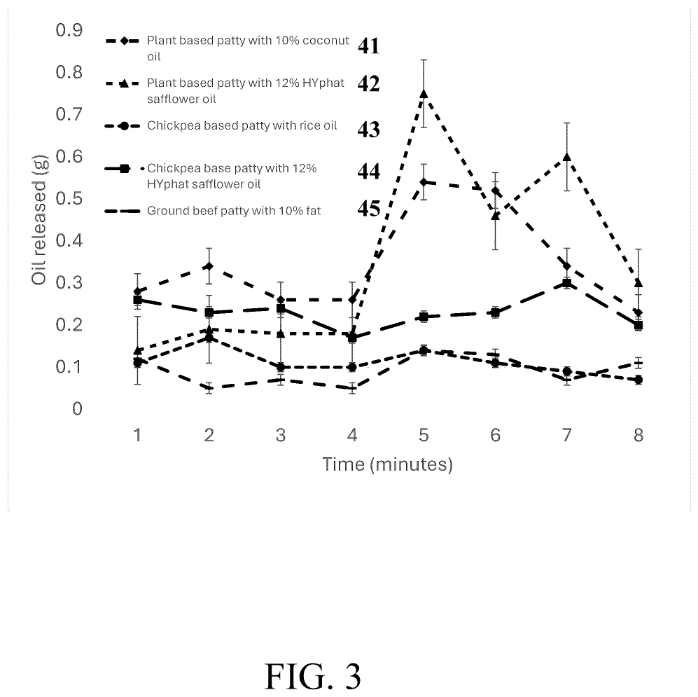

FIG. 3 illustrates various line graphs of the oil released over time from the fungal pellet with encapsulated plant oil incorporated into a plant-based burger food product. The x-axis shows time (minutes) and the y-axis shows grams of oil released. Each line connotates different products with or without fungal pellets. Measurements were taken by weighing oil released from the burger patty application over time as it was grilled on a skillet at 325° F. (162.8° C.) for eight minutes, turned over at 4 minutes, with measurements taken at one minute intervals. Line graph (41) shows experimental data from a plant based patty with 10% coconut oil, line graph (42) shows experimental data from a plant based patty with 12% fungal pellet with encapsulated safflower oil as the fat ingredient, line graph (43) shows experimental data from a chickpea based patty with rice oil as the fat ingredient, line graph (44) shows experimental data from a chickpea based patty with 12% fungal pellet with encapsulated safflower oil as the fat ingredient, and line graph (45) shows a ground beef patty with 10% endogenous animal fat as the fat ingredient.

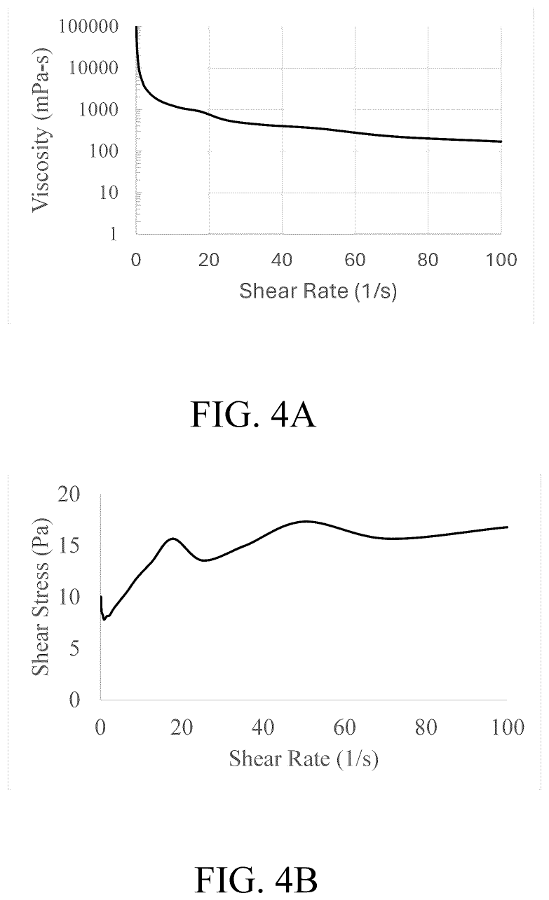

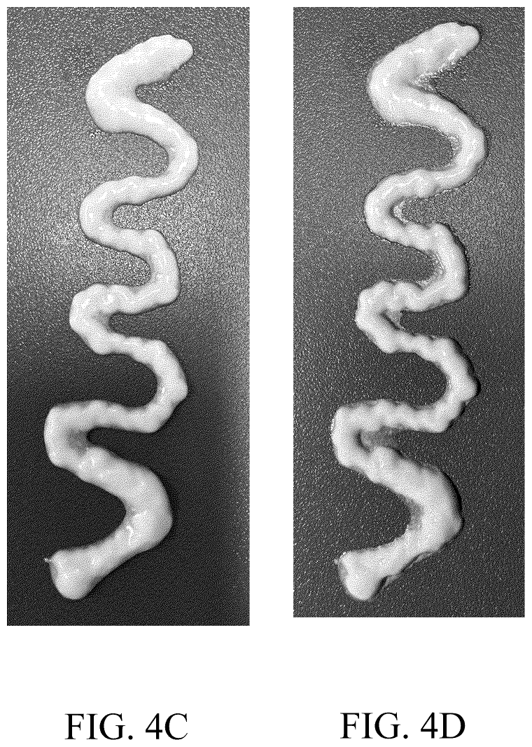

FIG. 4A shows experimental data in a graph relating viscosity (y-axis) as a function of shear rate (x-axis) for a stable emulsion prepared by mixing fungal pellets with water. The non-Newtonian nature of this emulsion is an unexpected finding. Measurements were collected by rheometer.

FIG. 4B Shows experimental data in a graph relating shear stress (y-axis) as a function of shear rate (x-axis) for a stable emulsion prepared by mixing fungal pellets with water. The non-Newtonian nature of this emulsion is an unexpected finding. Measurements were collected by rheometer.

FIG. 4C shows a photograph of an emulsion made from an oil encapsulated in fungal pellets extruded through a 3-4 mm opening of a syringe onto a tray at room temperature. The photograph was taken immediately after extrusion.

FIG. 4D is a picture of the emulsion described in FIG. 4C taken 2 hours later, with the emulsion remaining at room temperature for the 2 hours. Surprisingly, little to no degradation is seen over this two hour period demonstrating that a stable emulsion is generated.

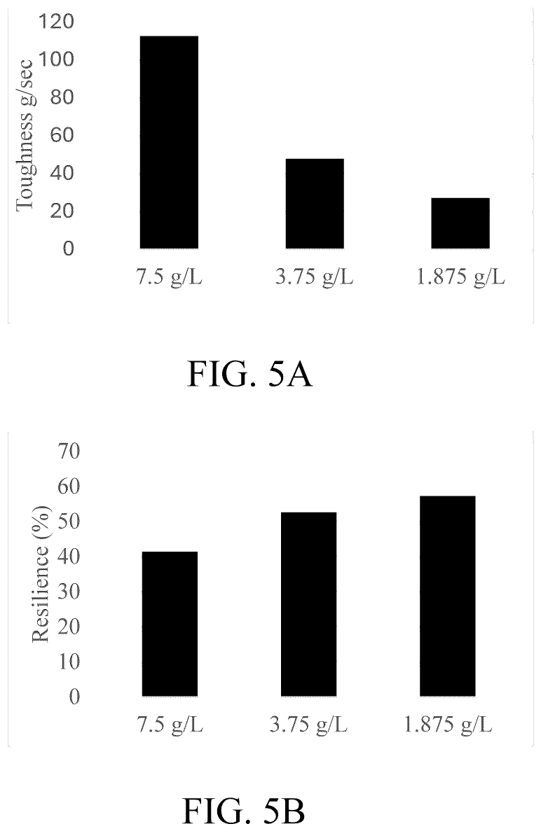

FIG. 5A shows a graph of experimentally determined values of toughness (g/sec) from a texture analyzer measuring fungal pellets cultivated under the same methodology with the exception of the growth media compositional concentration. The fungal pellets were grown in a complex media with approximately 7.5 g/L total sugar, a diluted complex media with 3.75 g/L total sugar, and a further diluted complex media at 1.875 g/L total sugar. This data was gathered using a Texture Analyzer from Stable Micro Systems using a flat probe.

FIG. 5B shows a graph of experimentally determined values of the % resilience from a texture analyzer measuring fungal pellets cultivated under the same methodology with the exception of the growth media compositional concentration. The fungal pellets were grown in a complex media with approximately 7.5 g/L total sugar, a diluted complex media with 3.75 g/L total sugar, and a further diluted complex media at 1.875 g/L total sugar. This data was gathered using a Texture Analyzer from Stable Micro Systems using a flat probe. The difference between the resilience measure as a function of media composition also gave an unexpected result.

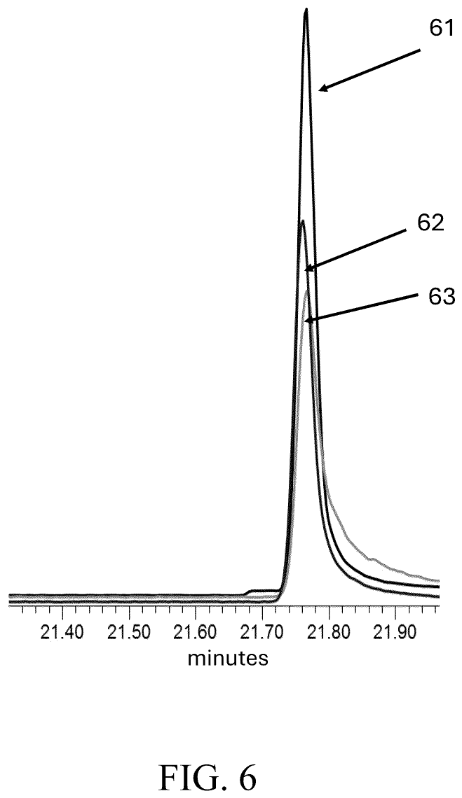

FIG. 6 shows an overlay of FID (flame ionization detector) gas chromatographs. Trace (61) identifies the trace of a muscone standard prepared at 10 wt % in methanol. Trace (62) identifies the positive identification of muscone based on the standard. Trace (61) results from a fungal pellet after cultivation in the presence of 20% (v/v) muscone that was rinsed with water thoroughly after separation from the muscone containing media and then prepared at a 10 wt % amount in methanol. Trace (63) identifies the positive identification of muscone based on the standard (61) from a fungal pellet after cultivation in the presence of 15% (v/v) muscone that was rinsed with water thoroughly after separation from the muscone containing media and prepared at 10 wt % in methanol. The concentration of muscone, a common fragrance compound, in cultivation media, was unexpectedly correlated with its encapsulation within a fungal pellet.

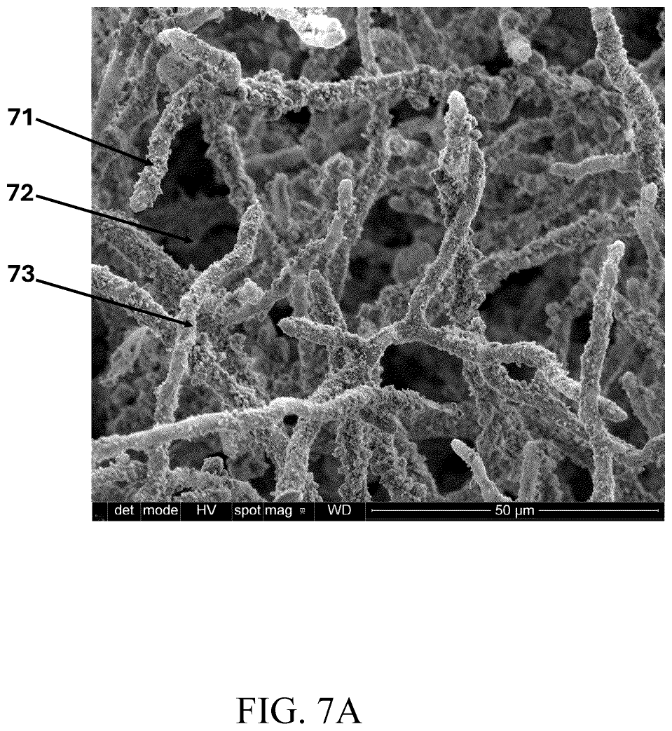

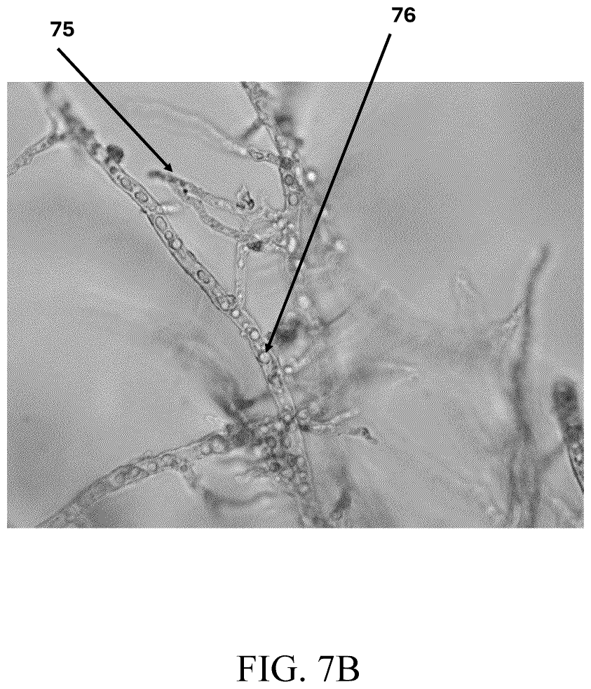

FIG. 7A shows a SEM image of a dehydrated fungal pellet with a hyphae strand (71), the component part of fungal pellets. The interstitial space (72) can be seen which can be loaded with fats and/or oils. Fats and/or oils (73) binding to the cell surface of the hyphae strands can be seen as an unexpected modification to the hyphae.

FIG. 7B is a photograph of a light microscope image taken with a 60× magnification lens of a fungal pellet. The hyphal strands (75) can be seen and the fats and/or oils (76) can be seen stabilized within the hyphal strand of the fungal pellet. This image was taken using the protocol of Nazir et al. 2022 & Burdon 1946 with appropriate modifications. This is a visual demonstration of the oil within the hyphae walls of the fungal pellet. The references are incorporated by reference for all purposes.

In an embodiment, the present invention relates to a composition comprising a fungal pellet, one or more fats and/or one or more oils, wherein the one or more fats and/or one or more oils are optionally from an exogenous source.

In a variation, the fungal pellet fats and/or oils are deposed in and/or on a mycelium hyphae and/or an interstitial space of the fungal pellet. In a variation, the composition optionally further comprises an emulsion of a fat.

In a variation, the fungal pellet further comprises an emulsion, and/or an oleogel, and/or a hydrocolloid comprising fats and/or oils, wherein one or more of the emulsion, oleogel, and/or hydrocolloid are absorbed into the interstitial space of the fungal pellet.

In a variation, the composition further comprises additional fats and/or oils in a mycelium of the fungal pellet, the mycelium having an interior first layer, an additional second layer, and optionally an outer third layer, wherein the additional second layer of the fungal pellet is exterior to the interior first layer. In a variation, the various layers have different oil and/or fat amounts and/or different compositions.

In a variation, the fungal pellet is encapsulated by a shell material that comprises one or more of wax, agar, alginate, chitin, protein polymer gel, and combinations thereof, In a variation, the shell generated by the shell material controls release of the one or more fats and/or the one or more oils.

In a variation, the composition further comprises one or more members selected from the group consisting of nutrients and emulsions, the nutrients and emulsions derived from raw agricultural goods, agricultural waste streams, and/or modified agricultural goods.

In a variation, a size of the fungal pellet is between about 50 microns and about 20 millimeters, and optionally, the composition comprises from about 10 wt % to about 90 wt % fats, based on a total dry weight of the fungal pellet, wherein a melting point of the fats is between 1° C. to 110° C.

In alternate embodiments, the physical parameters of the fungal pellets can exhibit a range of sizes from as small as 10 microns to as large as 100 millimeters. Alternatively, the fungal pellet size may range from 10-50 microns, or from 10-100 microns, or from 50-200 microns, or from 50-500 microns, or from 100 microns to 1 millimeter, or from 0.5-2 millimeters, or from 1-4 millimeters, or from 1-6 millimeters, or from 2-8 millimeters, or from 5-10 millimeters, or from 10-20 millimeters, or from 10-50 millimeters, or from 20-75 millimeters, or from 50-100 millimeters.

The fungal pellet may comprise from about 10 wt % to about 90 wt % fat, based on the total dry weight of the fungal pellet. Alternatively, the fat content may range from 10-20 wt %, or from 10-30 wt %, or from 10-40 wt %, or from 20-50 wt %, or from 30-60 wt %, or from 30-70 wt %, or from 40-80 wt %, or from 50-90 wt %.

The fat may be characterized by melting point ranges, including high melting point fats having a melting point from 36° C. to 110° C. Alternatively, the high melting point range may be from 36-50° C., or from 50-70° C., or from 70-90° C., or from 90-110° C. Low melting point fats may have a melting point from 1° C. to 36° C. Alternatively, the low melting point range may be from 1-10° C., or from 10-20° C., or from 20-30° C., or from 30-36° C.

In a variation, the one or more fats and/or the one or more oils comprise one or more of plant-based fat, natural animal fat, cell cultured animal fat, synthetic fat, triglycerides, triacylglycerols, caproic acid, caprylic acid, 12-hydroxystearic acid, 9-hydroxystearic acid, 10-hydroxystearic acid, castor oil, algal oil, microbial oil, fish oil, palm oil, palm kernel oil, rapeseed oil, sunflower oil, coconut oil, canola oil, soybean oil, flaxseed oil, wheat germ oil, corn oil, rice oil, olive oil, cottonseed oil, safflower oil, sesame oil, argan oil, walnut oil, almond oil, babassu oil, shea butter, shea kernel oil, mango butter, cocoa butter, borage oil, black currant oil, sea-buckthorn oil, macadamia oil, saw palmetto oil, rice bran oil, peanut oil, linolenic acid, gamma linolenic acid, alpha-linolenic acid, eicosapentaenoic acid, docosahexaenoic acid, stearidonic acid, or eicosatetraenoic acid and combinations thereof.

As an example, fungal pellets prepared as described above, with the addition of high oleic safflower oil has the fatty acid composition provided in Table 1, and Table 2.

TABLE 1
Fat composition of fungal pellet encapsulating
high-oleic safflower oil

| Analyte | Result (g/100 g) |
|---|---|
| Saturated FA | 6.416 |
| Monounsaturated FA | 59.664 |
| Polyunsaturated FA | 13.33 |
| Trans FA | 0.155 |
| Total Fatty Acids | 79.566 |
| Omega 3 | 1.418 |
| Omega 6 | 11.912 |
| Omega 9 | 58.619 |

TABLE 2
Fatty acid profile of fungal pellet encapsulating
high-oleic safflower oil

| Analyte | Result (g/100 g) |
|---|---|
| Caprylic | 0.011 |
| Capric | 0.0067 |
| Myristic | 0.0549 |
| Palmitic | 4.1487 |
| trans-Palmitoleic | 0.0459 |
| Palmitoleic | 0.1087 |
| Heptadecanoic | 0.0375 |
| Stearic | 1.3922 |
| Elaidic | 0.0116 |
| Oleic | 57.9548 |
| trans-12-Linolelaidic | 0.0488 |
| Linoleic | 11.8953 |
| trans-Linolenic | 0.0228 |
| a-Linolenic | 1.418 |
| Arachidic | 0.3469 |
| trans-Eicsenoic | 0.0261 |
| cis-11-Eicosenoic | 0.4186 |
| cis-11,14- | 0.017 |
| Eicosadienoic | |
| Behenic | 0.2332 |
| Erucic | 0.0201 |
| Tricosanoic | 0.0273 |
| Lignoceric | 0.149 |
| Nervonic | 0.1166 |

Table 3 shows the oil release ratio in percentage of various encapsulated fungal pellets.

TABLE 3
Oil release ratio of encapsulated pellets

| Media Composition | % released | Pellet/Oil Ratio |
|---|---|---|
| Potato dextrose broth media | 18.52 | 15.10 |
| Synethic complete media | 14.34 | 9.67 |
| with soybean oil | | |
| Almond Extract with soybean | 3.22 | 3.48 |
| oil | | |
| Rice extract with canola oil | 17.80 | 1.52 |
| Rice extract and brown color | 20.88 | 1.69 |
| with canola oil | | |
| Rice and tomato extract with | 10.53 | 2.56 |
| canola oil | | |
| Rice extract with flax seed oil | 15.08 | 1.30 |

Pellets loaded with safflower oil, cooked at 300° F. for 4 minutes

Table 4 shows encapsulation of oil in fungal pellets from a variety of fungal species.

TABLE 4

| | | Pellet/Oil Ratio | |
|---|---|---|---|
| Genus Species | Fat or Oil | by weight | Growth Media |
| Aspergillus oryzae | Safflower | 15.10 | Potato Dextrose |
| | | | Broth (PDB) |
| Aspergillus Awamori | Safflower | 12.05 | Potato Dextrose |
| | | | Broth (PDB) |
| Penicillium roqueforti | Safflower | 5.13 | Potato Dextrose |
| | | | Broth (PDB) |
| Auricularia auricula | Safflower | 8.72 | Potato Dextrose |
| | | | Broth (PDB) |

In a variation, the emulsion is derived from emulsifiers comprising one or more of a protein, a cellulose, a single glyceride, a double glyceride, lactylated glyceride, acylated glyceride, alkoxylated glyceride, a glyceride esters connected with diacetyl tartaric acid, phospholipids, lecithin, starch modified with succinic acid, modified corn starch, gum Arabic, gum Arabic derivatives with succinic acid, saponins from Quillaya, fatty acid salts of magnesium, fatty acid salts of potassium, fatty acid salts of calcium, polysorbates, fatty acid lactylates from alkaline earth metals, esters derived from sugars, sodium phosphates, sodium dodecyl sulfate, cetyl alcohol, stearyl alcohol, cetearyl alcohol, myristyl alcohol, behenyl alcohol, oleyl alcohol, lauryl alcohol, isostearyl alcohol, lanolin alcohol, arachidyl alcohol, and isocetyl alcohol, beeswax, or Guar Gum, or combinations thereof.

In a variation, the hydrocolloid material comprises one or more members selected from the group consisting of pullulan, alginate, cross linked alginate, sodium alginate, propylene glycol alginate, pectin, amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, and chitosan, or combinations thereof.

In a variation, the fungal pellet is derived from a fungus, said fungus being one or more members selected from the group consisting of Aspergillus sp., Penicillium sp., Agaricus sp., Amanita sp., Armillaria sp., Auricularia sp., Boletus sp., Bovista sp., Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp., or combinations thereof.

In a variation, the composition further comprises additives, the additives comprising one or more of flavoring compounds, coloring compounds, scented compounds, nutrients, minerals, vitamins, salts, peptides, collagen and combinations thereof.

In further aspects, the food flavorings that may be added are selected from the group consisting of vanilla extract, almond extract, cinnamon, nutmeg, ginger, clove, cardamom, anise, allspice, saffron, black pepper, white pepper, chili pepper, paprika, cumin, coriander, mustard, turmeric, lemongrass, basil, oregano, thyme, rosemary, sage, marjoram, parsley, dill, fennel, tarragon, bay leaf, mint, cilantro, lavender, chive, garlic, onion, smoked salt, hickory smoke, mesquite smoke, Worcestershire sauce, soy sauce, miso, tamari, tomato paste, mushroom extract, balsamic vinegar, apple cider vinegar, lemon juice, lime juice, orange zest, lemon zest, lime zest, pomegranate molasses, brown sugar, molasses, honey, maple syrup, liquid aminos, nutritional yeast, seaweed extract, kombu, bonito flakes, anchovy extract, butter flavor, cheese flavor, cream flavor, yogurt flavor, sour cream flavor, bacon flavor, ham flavor, sausage flavor, chicken flavor, roasted chicken flavor, turkey flavor, duck flavor, beef flavor, roasted beef flavor, veal flavor, lamb flavor, pork flavor, salmon flavor, tuna flavor, shrimp flavor, crab flavor, lobster flavor, oyster flavor, scallop flavor, umami flavor, roasted flavor, caramel flavor, lard flavor, tallow flavor, bone broth flavor, suet flavor, schmaltz flavor, fruit flavors (such as strawberry, raspberry, blueberry, banana, and pineapple), vanilla flavor, chocolate flavor, coffee flavor, cocoa flavor, caramelized onion flavor, roasted garlic flavor, truffle flavor, and a combination thereof.

In an embodiment, the present invention relates to a food product containing the composition as disclosed above.

In a variation, the food product is a burger patty, sausage, pepperoni, plant-based meat substitute, dairy alternative, baked good, snack, confectionery, dessert, processed food, gluten-free product, non-dairy whipped topping, or other food reliant on fats and/or oils for enhancing taste, texture, or sensory properties.

In a variation, the present invention relates to any of a plurality of methods including a method of encapsulating fats and/or oils in the mycelium hyphae of a fungal pellet, the method comprising: growing the fungal pellet in a media containing one or more fats and/or one or more oils the fats and/or oils comprising plant-based fats and/or oils, natural animal fats and/or oils, cell cultured animal fats and/or oils, or synthetic fats and/or oils.

In a variation of the method(s), a size of the fungal pellet is between 50 microns to 20 millimeters. In a variation, the method further comprises drying or freeze drying the fungal pellet.

In a variation, the method further comprises one or more steps of integrating fat flavor compounds during a growth of the fungal pellet to capture a taste, texture, and/or mouthfeel of traditional fats and/or oils, using an oleogel or oil and gums to create animal fats and/or oils tastes and textures, optimizing a surface to volume ratio absorption of fats and/or oils.

In a variation, the fats and/or oils are selected from the group consisting of vegetable oils, animal fats, nut oils, specialty oils, novel trans-fat replacements, butter, ghee, margarine, shortening formulations, specialty fats, modified fats, chemically modified fats, and cell-cultured fats and combinations thereof, wherein the fungal pellet comes from one or more fungi selected from the group consisting of Aspergillus sp., Penicillium sp., Agaricus sp., Amanita sp., Armillaria sp., Auricularia sp., Boletus sp., Bovista sp., Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp or combinations thereof.

In a variation of the method, the method may further comprise adding one or more materials selected from the group consisting of pullulan, alginate, cross linked alginate, sodium alginate, propylene glycol alginate, pectin, amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, and chitosan, or combinations thereof.

In a variation, the encapsulated fats and/or oils may be incorporated at different stages of fungal pellet growth to achieve a differential core-layer structure.

In a variation, the method may further comprise a step of adding additional fats and/or oils during post processing through water/oil exchange or drying and saturation with fats.

In a variation, the fats and/or oils that are encapsulated improve one or more of taste, texture, and/or flavor to alternative meat products, improve an appearance of alternative meat products, extend a release period of fats during a cooking process, improve a delivery of flavors incorporated into fats, provide a visually improved texture, aid in retaining fat during the cooking process, and/or deliver flavor during the cooking process.

In a variation, the method further comprises incorporating the fungal pellet(s) into a food product or into a cosmetic.

The following examples illustrate certain embodiments of the present invention. They are meant to be illustrative of how the present invention can be made and used but are not meant to constrain the breadth of the present invention.

### Example 1

The mycelium scaffold and fat encapsulation in hyphae Aspergillus oryzae (ATCC 46249) were grown on potato dextrose broth (PDB) agar plates for 2 days at 30° C. Aspergillus oryzae is inoculated into a 250 mL flask containing 120 mL of rice agricultural product emulsion with a standard pH of 6.1-6.4. Then cultured for 16-39 hours at 34-36° C. at 150 RPM to produce the final pellets at a size of about 3-6 mm.

In a variation, an alternative method can be employed in which pellets are prepared in the same manner and then the pellets are washed with sterile water, are pasteurized in water at 60° C. for 10 minutes, and are saturated in oil. Subsequently, the pellets can be dried or freeze dried by spreading pellets in a single layer on a tray and dried under vacuum with a drying temperature of 54° C. for 6-10 hours, after which the pellets can be stored at room temperature until further use.

In a variation, oil flavored with oil soluble flavor compounds can be added to the dry pellets at a ratio of 1.5-1.7 times the weight of the dry pellet. The finished pellet can be stored at 4° C. for long term storage, thereby providing a relatively long shelf life. Short term storage at room temperature can be accomplished without any adverse effects.

In a variation, an alternative method can be practiced in which pellets are prepared in the same manner as described above wherein a fat and/or oil coating is added to further encapsulate the fungal pellet.

### Example 2

A method for encapsulating additional ingredients during fermentation. Retinol solubilized in a sunflower oil matrix is added to the growth media at a concentrations of 0.2-0.6% wt immediately prior to an Aspergillus oryzae fungal pellet incubation. The spore culture is then added to the media and incubated at 34-36° C. at 150 RPM for a period of 12-32 hours. The resulting retinol encapsulated pellets are strained from the media through a 1-2 mm mesh sieve and collected for further processing into the targeted application.

In a variation, an alternative method can be employed in which pellets are prepared in the same manner and then the pellets are washed with sterile water, are pasteurized in water at 60° C. for 10 minutes. Subsequently, the pellets can be dried or freeze dried by spreading pellets in a single layer on a tray and dried under vacuum with a drying temperature of 54° C. for 6-10 hours, after which the pellets can be stored at room temperature until further use.

### Example 3

A method for producing a fungal pellet with multiple layers. An Aspergillus oryzae spore culture was incubated for 4-14 hours at 34-36° C. at 150 RPM in a rice agricultural product emulsion with canola oil. The resulting fungal pellets were strained through a 1-2 mm mesh screen and added to a second media composed of a rice agricultural product emulsion with polyunsaturated fatty acids derived from algae and incubated for 4-14 hours at 34-36° C. at 150 RPM to produce the final fungal pellets at a size of about 4-10 mm. The resulting fungal pellets are strained from the media through a 1-2 mm mesh screen and collected for further processing.

### Example 4

An emulsion was prepared by heating 280 mL of distilled water to 60-80° C. and heating 120 g of fat encapsulated mycelium pellets to 60-80° C. in a separate container. The fungal pellets were prepared by culturing a Aspergillus oryzae spore culture in a liquid complex media emulsion for 16-21 hours. After the resulting pellets were dried, additional oil was added to the interstitial space. The heated fungal pellets were added to the water phase at a homogenization speed of 2000 rpm and the homogenizer speed was subsequently increased to 3500 rpm. The resulting mixture was homogenized for an additional 5-10 minutes. Finally, the mixture was stirred until it cooled to 40° C. The emulsion mixture was stored at 4° C. for further processing into targeted application. In some embodiments the emulsion can be used as a yogurt alternative, cheese product alternative, and can be used in the spreads, dips, sauces, frostings, and schmears.

### Example 5

An Aspergillus oryzae spore culture was grown on potato dextrose broth (PDB) agar plates for 2 days at 30° C. Fungal spores were collected in sterile water, and inoculated into a 250 mL flask containing 120 mL of potato dextrose broth at a concentration of about 10,000 spores/ml. The liquid cultures were incubated with a complex media made from extracted agricultural byproducts and standardized to 20 g/L total sugars, 1.3 g/L nitrogen and trace minerals with a standard pH of 4.5-5.0 and cultured for 30-36 hours at 30-32° C. at 150 RPM to produce the final pellets at a size of about 1-3 mm.

In a variation, an alternative method can be employed in which pellets are prepared in the same manner and then the pellets are washed with sterile water, are pasteurized in water at 60° C. for 10 minutes. Subsequently, the pellets can be dried or freeze dried by spreading pellets in a single layer on a tray and dried under vacuum with a drying temperature of 54° C. for 6-10 hours, after which the pellets can be stored at room temperature until further use.

In a variation, a plant based oil can be added to the dry pellets at a ratio of 3-5 times the weight of the dry pellet. The finished pellet can be stored at 4° C. for long term storage, thereby providing a relatively long shelf life. Short term storage at room temperature can be accomplished without any adverse effects.

In a variation, an alternative method can be practiced in which pellets are prepared in the same manner as described about above wherein a fat and/or oil coating is added to further encapsulate the fungal pellet.

### Example 6

A plant based patty was made by mixing 12 grams red color with 468 grams water. The water was combined with 240 grams TVP protein powder and allowed to hydrate. A second mixture of 240 grams water, 112.5 grams safflower oil, and 22 grams methylcellulose were combined and blended for 5 minutes. The second mixture was mixed into the hydrated TVP in an electric stand mixer for 5 minutes and 22.5 grams of carrageenan, 18 grams of salt, 37.5 grams of natural beef flavor were added to the stand mixer and mixed for 10 minutes. 210 grams oil encapsulated fungal pellets that included oil soluble fat flavor components was added to the stand mixer and mixed for 5 minutes. The final blend was formed into patties and frozen for future use.

### Example 7

A method is disclosed for preparing a plant-based pepperoni analogue by hydrating textured vegetable protein (TVP) in an aqueous solution containing colorants in concentrations ranging from approximately 0.01% to 5% by weight. This hydrated TVP is then blended with an emulsion consisting of water, oil, and methylcellulose, wherein the emulsion has an oil content ranging from about 10% to 40%, forming a homogeneous mixture. To achieve the desired flavor and texture, flavor enhancers, carrageenan at 0.1% to 3%, salt at 0.5% to 2.5%, and encapsulated lactic acid in concentrations of about 0.1% to 1% are added. Oil-encapsulated Aspergillus oryzae fungal pellets containing oil-soluble fatty acids and smoky flavors are incorporated in concentrations of about 1% to 10%, further enhancing the taste profile.

The resultant mixture is then introduced into a cellulose-based edible casing, sealed, and refrigerated at temperatures temperature of 4° C., to facilitate flavor development and stabilization over a period of 12 to 72 hours. In an alternative embodiment, a plant-based salami analogue is produced by modifying the seasoning profile to include additional flavor components such as black pepper and garlic, and other flavors typically in concentrations of about 0.1% to 2%, imparting a distinct salami flavor. This variation follows the same casing and refrigeration processes as described above, yielding a versatile, plant-based meat analogue suitable for applications requiring pepperoni or salami alternatives with a realistic taste, texture, and mouthfeel.

### Example 8

A method is disclosed for creating a ground meat analogue utilizing a blend of Aspergillus oryzae fungal pellets with varied compositions to simulate the appearance, texture, and taste of ground meat. The formulation comprises a mixture of Aspergillus oryzae fungal pellets containing fat in a concentration ranging from approximately 1% to 30%, combined with coloring agents, such as red dye, in concentrations of about 0.01% to 5% by weight to achieve the desired visual appearance. Additionally, flavoring agents and nutrients are incorporated to enhance palatability and nutritional content. The fungal pellets possess diameters between approximately 0.5 mm and 10 mm, allowing for a varied mouthfeel. The blend may further include fat-loaded fungal pellets, containing oils in concentrations ranging from approximately 40% to 90%, designed to improve juiciness and provide a satisfying texture.

The fungal pellet mixture is bound together using an emulsion derived from fungal biomass, wherein the emulsion aids in cohesion and stability of the ground meat analogue. To facilitate gelation, the mixture is subjected to chilling at a temperature of 4° C., for a period of 2 to 12 hours. This chilling process enables the formation of a cohesive, meat-like texture in the final product.

The resulting fungal pellet-based ground meat analogue can be used as a direct replacement for animal-based ground meat in products such as patties, sausages, pepperoni, salami, and other processed meat analogues. The method disclosed provides a sustainable, plant-based alternative that achieves the desired texture, appearance, and sensory characteristics of conventional ground meat products.

### Example 9

An Auricularia auricula spore culture was grown on potato dextrose broth (PDB) agar plates for 6 days at 30° C. Fungal spores were collected in sterile water, and 2 mL was inoculated into a 250 mL flask containing 120 mL of potato dextrose broth at a pH of 4.9-5.2. The flasks were incubated at 30° C. and 150 RPM for 24 hours. 3 mL of the media and fungal mass was collected from the resulting flask and incubated at 30° C. for 120 hours.

In a variation, an alternative method can be employed in which pellets are prepared in the same manner and then the pellets are washed with sterile water, are pasteurized in water at 60° C. for 10 minutes. Subsequently, the pellets can be dried or freeze dried by spreading pellets in a single layer on a tray and dried under vacuum with a drying temperature of 54° C. for 6-10 hours, after which the pellets can be stored at room temperature until further use.

In a variation, a plant based oil can be added to the dry pellets at a ratio of 1-3 times the weight of the dry pellet. The finished pellet can be stored at 4° C. for long term storage, thereby providing a relatively long shelf life. Short term storage at room temperature can be accomplished without any adverse effects.

### Example 10

An Aspergillus awamori spore culture was grown on potato dextrose broth (PDB) agar plates for 5 days at 30° C. Fungal spores were collected in sterile water, and inoculated into a 250 mL flask containing 120 mL of carrot byproduct extracted media at a concentration of about 10,000 spores/ml at pH of 4.9-5.1. The flasks were incubated at 30° C. and 150 RPM for 480 hours. The resulting fungal pellets are strained from the media through a 1-2 mm mesh screen and collected for further processing.

### Example 11

An Aspergillus oryzae spore culture was grown on potato dextrose broth (PDB) agar plates for 3 days at 28° C. Fungal spores were collected in sterile water and inoculated into a 30 mL flask containing 150 mL of rice waste extract media at a concentration of about 10,000 spores/ml at pH of 4.2-4.4. The flasks were incubated at 28° C. and 160 RPM for 40 hours.

### Example 12

A Penicillium roqueforti spore culture was grown on potato dextrose broth (PDB) agar plates for 6 days at 25° C. Fungal spores were collected in sterile water and inoculated into a 250 mL flask containing 100 mL of corn stillage media at a concentration of about 10,000 spores/ml at pH of 5.5-5.7. The flasks were incubated at 25° C. and 140 RPM for 48 hours.

### Example 13

An Auricularia auricula spore culture was grown on potato dextrose broth (PDB) agar plates for 4 days at 27° C. Fungal spores were collected in sterile water and inoculated into a 250 mL flask containing 130 mL of potato waste media at a concentration of about 10,000 spores/ml at pH of 4.8-5.0. The flasks were incubated at 27° C. and 145 RPM for 48 hours.

### Example 14

An Aspergillus awamori spore culture was grown on potato dextrose broth (PDB) agar plates for 5 days at 32° C. Fungal spores were collected in sterile water and inoculated into a 250 mL flask containing 110 mL of sweet potato waste media at a concentration of about 10,000 spores/ml at pH of 5.0-5.2. The flasks were incubated at 32° C. and 155 RPM for 50 hours.

### Example 15

An Aspergillus oryzae spore culture was grown on potato dextrose broth (PDB) agar plates for 2 days at 35° C. Fungal spores were collected in sterile water and inoculated into a 250 mL flask containing 140 mL of rice bran extract media at a concentration of about 10,000 spores/ml at pH of 4.5-4.7. The flasks were incubated at 35° C. and 135 RPM for 42 hours.

### Example 16

A Penicillium roqueforti spore culture was grown on potato dextrose broth (PDB) agar plates for 4 days at 26° C. Fungal spores were collected in sterile water and inoculated into a 250 mL flask containing 125 mL of apple pomace media at a concentration of about 10,000 spores/ml at pH of 4.6-4.8. The flasks were incubated at 26° C. and 145 RPM for 48 hours.

### Example 17

An Auricularia auricula spore culture was grown on potato dextrose broth (PDB) agar plates for 3 days at 34° C. Fungal spores were collected in sterile water and inoculated into a 250 mL flask containing 115 mL of carrot waste media at a concentration of about 10,000 spores/ml at pH of 5.3-5.5. The flasks were incubated at 34° C. and 130 RPM for 42 hours.

### Example 18

An Aspergillus Awamori spore culture was grown on potato dextrose broth (PDB) agar plates for 5 days at 29° C. Fungal spores were collected in sterile water and inoculated into a 250 mL flask containing 135 mL of almond hull extract media at a concentration of about 10,000 spores/ml at pH of 5.8-6.0. The flasks were incubated at 29° C. and 150 RPM for 45 hours.

### Example 19

An Aspergillus oryzae spore culture was grown on potato dextrose broth (PDB) agar plates for 2 days at 33° C. Fungal spores were collected in sterile water and inoculated into a 250 mL flask containing 140 mL of barley byproduct media at a concentration of about 10,000 spores/ml at pH of 4.4-4.6. The flasks were incubated at 33° C. and 135 RPM for 40 hours.

### Example 20

A Penicillium roqueforti spore culture was grown on potato dextrose broth (PDB) agar plates for 6 days at 27° C. Fungal spores were collected in sterile water and inoculated into a 250 mL flask containing 110 mL of potato waste media at a concentration of about 10,000 spores/ml at pH of 5.1-5.3. The flasks were incubated at 27° C. and 160 RPM for 50 hours.

### Example 21

An Auricularia auricula spore culture was grown on potato dextrose broth (PDB) agar plates for 4 days at 31° C. Fungal spores were collected in sterile water and inoculated into a 250 mL flask containing 120 mL of walnut extract media at a concentration of about 10,000 spores/ml at pH of 4.7-4.9. The flasks were incubated at 31° C. and 140 RPM for 30 hours.

### Example 22

An Aspergillus awamori spore culture was grown on potato dextrose broth (PDB) agar plates for 5 days at 28° C. Fungal spores were collected in sterile water and inoculated into a 250 mL flask containing 100 mL of rice waste media at a concentration of about 10,000 spores/ml at pH of 5.4-5.6. The flasks were incubated at 28° C. and 150 RPM for 40 hours.

### Example 23

An Aspergillus oryzae spore culture was grown on potato dextrose broth (PDB) agar plates for 3 days at 35° C. Fungal spores were collected in sterile water and inoculated into a 250 mL flask containing 130 mL of potato waste media at a concentration of about 10,000 spores/ml at pH of 5. The flasks were incubated at 35° C. and 150 RPM for 18 hours.

### Example 24

A Penicillium roqueforti spore culture was grown on potato dextrose broth (PDB) agar plates for 5 days at 25° C. Fungal spores were collected in sterile water and inoculated into a 250 mL flask containing 105 mL of tomato waste media at a concentration of about 10,000 spores/ml at pH of 5.2-5.4. The flasks were incubated at 25° C. and 155 RPM for 30 hours.

### Example 25

An Auricularia auricula spore culture was grown on potato dextrose broth (PDB) agar plates for 2 days at 32° C. Fungal spores were collected in sterile water and inoculated into a 250 mL flask containing 145 mL of sugar potato waste media at a concentration of about 10,000 spores/ml at pH of 4.5-4.7. The flasks were incubated at 32° C. and 150 RPM for 47 hours.

It should be understood and it is contemplated and within the scope of the present invention that any feature that is enumerated above can be combined with any other feature that is enumerated even if they do not appear together as long as those features are not incompatible. Whenever ranges are mentioned, any real number that fits within the range of that range is contemplated as an endpoint to generate subranges. In any event, the invention is defined by the below claims.

We claim:

1. A composition comprising a fungal pellet, one or more fats and/or one or more oils, wherein the one or more fats and/or one or more oils are optionally from an exogenous source.

2. The composition of claim 1, wherein the fungal pellet fats and/or oils are deposed in and/or on a mycelium hyphae and/or an interstitial space of the fungal pellet, wherein the composition optionally further comprises an emulsion of a fat.

3. The composition of claim 2, wherein the fungal pellet further comprises an emulsion, and/or an oleogel, and/or a hydrocolloid comprising fats and/or oils, wherein one or more of the emulsion, oleogel, and/or hydrocolloid are absorbed into the interstitial space of the fungal pellet.

4. The composition of claim 1, further comprising additional fats and/or oils in a mycelium of the fungal pellet, the mycelium having an interior first layer, an additional second layer, and optionally an outer third layer, wherein the additional second layer of the fungal pellet is exterior to the interior first layer.

5. The composition of claim 1, wherein the fungal pellet is encapsulated by a shell material that comprises one or more of wax, agar, alginate, chitin, protein polymer gel, and combinations thereof, wherein the shell controls release of the one or more fats and/or the one or more oils.

6. The composition of claim 1, wherein the composition further comprises one or more members selected from the group consisting of nutrients and emulsions, the nutrients and emulsions derived from raw agricultural goods, agricultural waste streams, and/or modified agricultural goods.

7. The composition of any one of claim 1, wherein a size of the fungal pellet is between about 50 microns and about 20 millimeters, and optionally, wherein the composition comprises from about 10 wt % to about 90 wt % fats, based on a total dry weight of the fungal pellet, wherein a melting point of the fats is between 1° C. to 110° C.

8. The composition of claim 3, wherein the one or more fats and/or the one or more oils comprise one or more of plant-based fat, natural animal fat, cell cultured animal fat, synthetic fat, triglycerides, triacylglycerols, caproic acid, caprylic acid, 12-hydroxystearic acid, 9-hydroxystearic acid, 10-hydroxystearic acid, castor oil, algal oil, microbial oil, fish oil, palm oil, palm kernel oil, rapeseed oil, sunflower oil, coconut oil, canola oil, soybean oil, flaxseed oil, wheat germ oil, corn oil, rice oil, olive oil, cottonseed oil, safflower oil, sesame oil, argan oil, walnut oil, almond oil, babassu oil, shea butter, shea kernel oil, mango butter, cocoa butter, borage oil, black currant oil, sea-buckthorn oil, macadamia oil, saw palmetto oil, rice bran oil, peanut oil, linolenic acid, gamma linolenic acid, alpha-linolenic acid, eicosapentaenoic acid, docosahexaenoic acid, stearidonic acid, or eicosatetraenoic acid and combinations thereof, and wherein the emulsion is derived from emulsifiers comprising one or more of a protein, a cellulose, a single glyceride, a double glyceride, lactylated glyceride, acylated glyceride, alkoxylated glyceride, a glyceride esters connected with diacetyl tartaric acid, phospholipids, lecithin, starch modified with succinic acid, modified corn starch, gum Arabic, gum Arabic derivatives with succinic acid, saponins from Quillaya, fatty acid salts of magnesium, fatty acid salts of potassium, fatty acid salts of calcium, polysorbates, fatty acid lactylates from alkaline earth metals, esters derived from sugars, sodium phosphates, sodium dodecyl sulfate, cetyl alcohol, stearyl alcohol, cetearyl alcohol, myristyl alcohol, behenyl alcohol, oleyl alcohol, lauryl alcohol, isostearyl alcohol, lanolin alcohol, arachidyl alcohol, and isocetyl alcohol, beeswax, or Guar Gum, or combinations thereof; and
wherein the hydrocolloid material comprises one or more members selected from the group consisting of pullulan, alginate, cross linked alginate, sodium alginate, propylene glycol alginate, pectin, amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, and chitosan, or combinations thereof, and
wherein the fungal pellet is derived from a fungus, said fungus being one or more members selected from the group consisting of Aspergillus sp., Penicillium sp., Agaricus sp., Amanita sp., Armillaria sp., Auricularia sp., Boletus sp., Bovista sp, Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp., or combinations thereof.

9. The composition of claim 1, further comprising additives, the additives comprising one or more of flavoring compounds, colored compounds, scented compounds, nutrients, minerals, vitamins, salts, peptides, collagen and combinations thereof.

10. A food product containing the composition of claim 1.

11. The food product of claim 10, wherein the food product is a burger patty, sausage, pepperoni, plant-based meat substitute, dairy alternative, baked good, snack, confectionery, dessert, processed food, gluten-free product, non-dairy whipped topping, or other food reliant on fats and/or oils for enhancing taste, texture, or sensory properties.

12. A method of encapsulating fats and/or oils in the mycelium hyphae of a fungal pellet, the method comprising: growing the fungal pellet in a media containing one or more fats and/or one or more oils the fats and/or oils comprising plant-based fats and/or oils, natural animal fats and/or oils, cell cultured animal fats and/or oils, or synthetic fats and/or oils.

13. The method of claim 12, wherein a size of the fungal pellet is between 50 microns to 20 millimeters.

14. The method of claim 12, wherein the method further comprises drying or freeze drying the fungal pellet.

15. The method of claim 12, wherein the method further comprises one or more steps of integrating fat flavor compounds during a growth of the fungal pellet to capture a taste, texture, and/or mouthfeel of traditional fats and/or oils, using an oleogel or oil and gums to create animal fats and/or oils tastes and textures, optimizing a surface to volume ratio absorption of fats and/or oils.

16. The method of claim 12, wherein the fats and/or oils are selected from the group consisting of vegetable oils, animal fats, nut oils, specialty oils, novel trans-fat replacements, butter, ghee, margarine, shortening formulations, specialty fats, modified fats, chemically modified fats, and cell-cultured fats and combinations thereof, wherein the fungal pellet comes from one or more fungi selected from the group consisting of Aspergillus sp., Penicillium sp., Agaricus sp., Amanita sp., Armillaria sp., Auricularia sp., Boletus sp., Bovista sp., Calbovista sp., Calvatia sp., Cantharellus sp., Chlorophyll um sp., Clitocybe sp., Clitopilus sp., Coprinus sp., Cortinarius sp., Craterellus sp., Entoloma sp., Flammulina sp., Fusarium sp., Gomphus sp., Grifola sp., Polypilus sp., Gyromitra sp., Helvella sp., Hericium sp., Hydnum sp., Hygrophorus sp., Lactarius sp., Leccinum sp., Lentinus sp., Lepiota sp., Chlorophyllum sp., Lepiota sp., Lepista sp., Clitocybe sp., Lycoperdon sp., Neurospora sp., Marasmius sp., Morchella sp., Phlogiotis sp., Pholiota sp., Pleurocybella sp., Pleurotus sp., Pluteus sp., Polypilus sp., Grifola sp., Polyozellus sp., Polyporus sp., Ramaria sp., Rozites sp., Russula sp., Sparassis sp., Strobilomyces sp., Stropharia sp., Suillus sp., Terfezia sp., Tremella sp., Tricholoma sp., Tuber sp., Volvariella sp., and Rhizopus sp or combinations thereof.

17. The method of claim 12, further comprising adding one or more materials selected from the group consisting of pullulan, alginate, cross linked alginate, sodium alginate, propylene glycol alginate, pectin amylopectin, methoxyl pectin, inulin, carrageenan, cellulose gum, xanthan gum, locust bean gum, gellan gum, guar gum, tara gum, konjac gum, poly(styrenesulfonate), polyglutamic acid, alginic acid, poly(acrylic acid), poly(aspartic acid), poly(glutaric acid), dextransulfate, carboxymethylcellulose, modified carboxymethylcellulose, hyaluronic acid, chondroitin sulfate, heparin, methylcellulose, hydroxypropylmethyl cellulose, hydroxypropyl cellulose, polyvinyl pyrrolidone, maltodextrin, polydextrose, modified starch, silica, tragacanth, gum acacia, modified gum acacia, xanthan gum alginate, agarose, gelatin, gelatin B, inulin, chitin, and chitosan, or combinations thereof, and
wherein optionally the encapsulated fats and/or oils are incorporated at different stages of fungal pellet growth to achieve a differential core-layer structure.

18. The method of claim 12, further comprising a step of adding additional fats and/or oils during post processing through water/oil exchange or drying and saturation with fats.

19. The method of claim 12, wherein the fats and/or oils that are encapsulated improve one or more of taste, texture, and/or flavor to alternative meat products, improve an appearance of alternative meat products, extend a release period of fats during a cooking process, improve a delivery of flavors incorporated into fats, provide a visually improved texture, aid in retaining fat during the cooking process, and/or deliver flavor during the cooking process.

20. The method of claim 12, wherein the method further comprises incorporating the fungal pellet into a food product or into a cosmetic.
